(19) United States
(12) Patent Application Publication
(10) Pub. No.: US 2025/0295701 A1
(43) Pub. Date: Sep. 25, 2025

(54) Kit for Induction of Continuous Expansion of Hepatoblast Organoids and/or Hepatocyte Organoids Under 3D Suspension Conditions and Use Thereof

(71) Applicant: South China University of Technology, Guangzhou, Guangdong (CN)

(72) Inventors: Yuyou DUAN, Guangzhou, Guangdong (CN); Haibin WU, Guangzhou, Guangdong (CN); Honglin CHEN, Guangzhou, Guangdong (CN)

(73) Assignee: South China University of Technology, Guangzhou, Guangdong (CN)

(21) Appl. No.: 18/857,809

(22) PCT Filed: Dec. 27, 2022
(86) PCT No.: PCT/CN2022/142420

§ 371 Date: Jun. 12, 2025

(30) Foreign Application Priority Data
Dec. 23, 2022 (CN) 202211668145X

(51) Int. Cl. A61K 35/407; A61P 1/16; C12N 5/071

(52) U.S. Cl. CPC A61K 35/407; A61P 1/16; C12N 5/0671; C12N 2501/119; C12N 2501/12; C12N 2501/155; C12N 2501/16; C12N 2501/33; C12N 2513/00; C12N 2533/90

(57) ABSTRACT

The present disclosure relates to a kit for induction of continuous expansion of hepatoblast organoids and/or hepatocyte organoids under 3D suspension conditions and use thereof. In the kit, the type of cytokines and the amount thereof are reduced, and Matrigel with low concentration is used for the culture and differentiation of organoids, which can support long-term maintenance and expansion of hepatoblast organoids and/or hepatocyte organoids more efficiently. In the presence of Matrigel, hepatocytes in mature hepatocyte organoids are in polarization state that is similar to those of in vivo liver tissues and are obviously different from non-polarized hepatocyte spheroids under 3D suspension culture condition in terms of structure. Using the kit can allow the realization of large-scale culture and expansion of organoids in vitro, which meets the needs on functional hepatocytes in clinical treatments, drug screening and development, and other fields at aspects of cell yield, function, costs.

### CROSS-REFERENCE TO RELATED APPLICATIONS

The present application is a national phase entry under 35 USC § 371 of International Application PCT/CN2022/142420, filed Dec. 27, 2022, which claims the benefit of and priority to Chinese Patent Application No. 202211668145.X, filed Dec. 23, 2022, the entire disclosures of which are incorporated herein by reference.

### TECHNICAL FIELD

The present disclosure belongs to the field of biotechnologies, and particularly, to a kit for the induction of continuous expansion of hepatoblast organoids and/or hepatocyte organoids under 3D suspension conditions and use thereof.

### BACKGROUND

With multidirectional differentiation potential and unlimited self-renewal ability, human pluripotent stem cells (hPSCs) are widely used in cell therapy, drug development and other fields. In clinical practice, for patients with acute liver failure or end-stage liver disease, the best treatment is liver organ transplantation. However, due to the shortage of donor liver supply, patients are often unable to wait for a suitable donor liver for transplantation surgery, thus losing the best opportunity for the treatment. Therefore, human hepatocyte transplantation therapy and bioartificial liver treatment have become alternative options. However, clinical hepatocyte transplantation or bioartificial liver treatment for patients with liver diseases requires 10¹⁰ (10 billion) mature hepatocytes. The primary hepatocytes are scarce in source and difficult to expand, culture and maintain function under in vitro culture conditions, thus unable to meet the needs of clinical treatments. Therefore, the main challenge now is to obtain functional and transplantable hepatocytes in large scale to meet the growing demand for clinical treatment and drug development. Because hPSCs have characteristics of multidirectional differentiation potential and unlimited self-renewal ability, hPSCs can serve as seed cells to continuously differentiate into hepatocytes in vitro, thus providing the possibility to solve the shortage of hepatocyte supply.

In recent years, researchers have conducted in-depth research in large-scale culture of hPSCs and developed a variety of hPSCs large-scale culture methods. However, due to requiring the long time to induce PSCs to differentiate towards hepatocytes, the medium and cytokines consumed during the culture and differentiation are too expensive, and there are also differences among batches in each differentiation, thus it is difficult to produce 10¹⁰ or more PSCs and then use them to perform hepatocyte differentiation. The liver has the ability to regenerate in vivo. For example, for mouse liver, even after two-thirds of the liver is removed during the hepatectomy, it can regenerate a complete liver within a few weeks, mainly due to that liver progenitor cells in the liver play major roles in liver regeneration. Therefore, researchers can achieve the purpose of large-scale production of functional hepatocytes by obtaining hepatoblasts (liver progenitor cells)in vitro and inducing long-term culture of hepatoblasts. However, due to the scarcity of donor livers, obtaining hepatoblasts from the donor livers or inducing primary hepatocytes into hepatoblasts will limit their production capacity, thus leading to the number of hepatoblasts which can be expanded in vitro is also limited. Therefore, differentiating from hPSCs with unlimited proliferation ability into hepatoblasts that can be continuously expanded for a long time and then differentiating the expanded hepatoblasts into mature hepatocytes will be an appropriate approach to meet the clinical and biopharmaceutical needs on hepatocytes in terms of function, yield and cost.

Currently, most researchers have studied the differentiation of hPSCs into hepatoblasts and carried out long-term culture under 2D adherent culture conditions. However, 2D differentiation system cannot simulate 3D microenvironment of human liver. Secondly, increasing evidences exhibit that 3D culture system can better simulate the in vivo microenvironment and promote the generation of cells with liver lineage and hepatocyte maturation. In addition, the development of the organoids better mimics the structure and function of tissues and organs. However, the current development and culture of the organoids are based on high concentration of Matrigel, which seriously limits the large-scale production and expansion of the organoids.

Researchers have developed a method for the long-term culture of liver progenitor cells differentiated from PSCs under 2D culture conditions, and these hepatoblasts can be expanded for over 10 passages under 2D culture conditions in vitro and can be induced into hepatocytes with certain liver functions, and eventually a large number of hepatocytes can be obtained for the purpose of being used for biopharmaceutical research and the treatment with bioartificial liver device^{[1, 2]}. Some researchers have also developed a method for the long-term culture of hepatoblast organoids differentiated from hPSCs under 2D culture conditions. Hepatoblast organoids are expanded for 20 passages under 2D culture conditions in vitro, and can be induced into mature hepatocytes, which are intended to be used for drug screening and research on various liver diseases^{[1, 2]}.

However, due to 2D culture differentiation system cannot simulate three-dimensional microenvironment of human liver, hepatocytes obtained through this differentiation system have low maturity and incomplete functions. In addition, 2D culture conditions are not conducive to large-scale production of hepatoblasts or hepatoblast organoids, because researchers need to use cell factories or a kind of bioreactors for cell adherent growth for large-scale production under 2D culture conditions.

However, using these methods to obtain a large number of cells consumes a lot of labour-power and material resources, and the costs are too expensive, which limits their applications. Furthermore, various organoids, including hepatic organoids, currently developed under high-concentration Matrigel culture conditions, is not suitable for large-scale preparation and the costs is more higher, making them unsuitable for clinical translational research and applications^{[3,4]}.
1. Pan, T., et al., Robust expansion and functional maturation of human hepatoblasts by chemical strategy. Stem Cell Res her, 2021. 12(1): p. 151.
2. Chen, S., et al., Hepatic spheroids derived from human pluripotent stem cells in bio-artificial liver induced porcine acute hepatic failure. Cell Res, 2020. 30(1): p. 95-97.
3. Hu, H., et al., Long-Term Expansion of Functional Mouse and Human Hepatocytes as 3D Organoids. Cell, 2018. 175(6): p. 1591-1606.e19.
4. Wang, S., et al., Human ESC-derived expandable hepatic organoids enable therapeutic liver repopulation and pathophysiological modeling of alcoholic liver injury. Cell Res, 2019. 29(12): p. 1009-1026.

In conclusion, the shortcomings of prior art include: 1. The differentiation time course is longer, thus the time cost is high. Since 2D culture the differentiation system can simulate the developmental process of hepatocytes, but cannot simulate the three-dimensional microenvironment of human liver, thus it takes longer time to obtain hepatocytes with same maturity through this differentiation system, furthermore, it is difficult to obtain mature and fully functional hepatocytes which are generated in the three-dimensional microenvironment of human liver. 2. Many expensive cell growth factors are used for the differentiation at higher concentrations, thus the costs of reagents and material consumed are high. 3. The maturity and function of hepatocytes obtained through the differentiation of human pluripotent stem cells need to be further improved. 4. Using high-concentration Matrigel to differentiate and culture the organoids is costly. 5. The entire differentiation and culture process have many steps, and the manipulation of the process is complex.

Therefore, how to reduce cytokines and their dosage and use low concentrations of Matrigel to differentiate and culture the organoids as well as more efficiently support long-term maintenance and expansion of hepatoblast organoids are issues to be solved urgently in the art.

### SUMMARY

The object of first aspect of present disclosure is to provide a medium composition.

The object of second aspect of present disclosure is to provide a kit.

The object of third aspect of present disclosure is to provide uses of the above-mentioned medium composition and kit.

The object of fourth aspect of present disclosure is to provide a method for developing and/or continuously expanding hepatoblast organoids under 3D suspension conditions.

The object of fifth aspect of present disclosure is to provide a method for developing mature hepatocyte organoids under 3D suspension conditions.

The object of sixth aspect of present disclosure is to provide the uses of hepatoblast organoids and hepatocyte organoids developed by the above methods under 3D suspension conditions.

The technical solutions used by present disclosure are as follows:

According to the first aspect of present disclosure, a medium composition including the fifth medium is provided, and the fifth medium comprises Matrigel, a TGFβ/ALK inhibitor, a GSK3β inhibitor, and Forskolin (FSK).

Preferably, a final concentration of the Matrigel in the fifth medium is 3-8 v/v %.

Preferably, the final concentration of the Matrigel in the fifth medium is 4-7 v/v %.

Preferably, the final concentration of the Matrigel in the fifth medium is 5 v/v %.

Preferably, the TGFβ/ALK inhibitor in the fifth medium comprises at least one of SB431542, SB-505, A-83-01, GW6604, IN-1130, Ki26894, LY2157299, LY364947 (HTS-466284), LY550410, LY573636, LY580276, NPC-30345, SB-505124, SD-093, Sm16, SM305, SX-007, Antp-Sm2A, and LY2109761.

More preferably, the TGFβ/ALK inhibitor in the fifth medium comprises SB431542.

Preferably, the GSK-3 inhibitor in the fifth medium comprises at least one of B216763, TWS119, NP031112, SB216763, CHIR-98014, AZD2858, AZD1080, SB415286, LY2090314, and CHIR-99021.

More preferably, the GSK-3 inhibitor in the fifth medium comprises CHIR-99021.

Preferably, the fifth medium also comprises growth factors and Bone morphogenetic protein (BMP) signaling pathway activators.

Preferably, the BMP signaling pathway activators in the fifth medium comprise at least one of BMP2, BMP4, SB4, SJ000291942, SJ000063181, SJ000370178, isoliquiritigenin, diosmetin, apigenin and biochanin.

More preferably, the BMP signaling pathway activators in the fifth medium comprise BMP4.

Preferably, the growth factors in the fifth medium comprise at least one of epidermal growth factor (EGF), platelet-derived growth factor (PDGF), fibroblast growth factor (FGF), hepatocyte growth factor (HGF), insulin-like growth factor-I (IGF-1), IGF-II, leukaemia inhibitory factor (LIF), nerve growth factor (NGF), oncostatin M (OSM), platelet-derived endothelial cell growth factor (PDECGF), transforming growth factor-α (TGF-α) and vascular endothelial cell growth factor (VEGF).

More preferably, the growth factors in the fifth medium comprise epidermal growth factor (EGF) and fibroblast growth factor (FGF); and fibroblast growth factor (FGF) is preferably fibroblast growth factor 4 (FGF-4).

Preferably, the fifth medium also comprises FBS, ITS, NEAA, GlutaMAX, and nicotinamide.

Preferably, a final concentration of the TGFβ/ALK inhibitor in the fifth medium is 5-20 μM.

Preferably, a final concentration of the GSK3β inhibitor in the fifth medium is 3-8 μM.

Preferably, a final concentration of FSK in the fifth medium is 5-20 μM.

Preferably, a final concentration of the epidermal growth factor (EGF) in the fifth medium is 10-30 ng/mL.

Preferably, a final concentration of the fibroblast growth factor 4 (FGF-4) in the fifth medium is 10-30 ng/mL.

Preferably, a final concentration of the BMP signaling pathway activator in the fifth medium is 10-30 ng/mL.

Preferably, a final concentration of FBS in the fifth medium is 5-15 w/w %.

Preferably, a final concentration of ITS in the fifth medium is 0.5-1.5 w/w %.

Preferably, a final concentration of glutamax in the fifth medium is 0.5-1.5 w/w %.

Preferably, a final concentration of NEAA in the fifth medium is 0.5-1.5 w/w %.

Preferably, a final concentration of the nicotinamide in the fifth medium is 5-15 mM.

Preferably, basal medium of the fifth medium is at least one of RPMI1640 medium and IMDM medium.

Preferably, the fifth medium is used to induce and culture the differentiated hepatoblasts into hepatoblast organoids, and to maintain sustainable culture of hepatoblast organoids.

Preferably, the medium composition further includes a sixth medium, and the sixth medium comprises Matrigel and growth factors.

Preferably, a final concentration of the Matrigel in the sixth medium is 3-8 v/v %.

Preferably, the final concentration of the Matrigel in the sixth medium is 4-7 v/v %.

Preferably, the final concentration of the Matrigel in the sixth medium is 5 v/v %.

Preferably, the growth factors in the sixth medium comprise at least one of epidermal growth factor (EGF), platelet-derived growth factor (PDGF), fibroblast growth factor (FGF), hepatocyte growth factor (HGF), insulin-like growth factor-I (IGF-1), IGF-II, leukaemia inhibitory factor (LIF), nerve growth factor (NGF), oncostatin M (OSM), platelet-derived endothelial cell growth factor (PDECGF), transforming growth factor-α (TGF-α) and vascular endothelial cell growth factor (VEGF).

More preferably, the growth factors in the sixth medium comprise hepatocyte growth factor (HGF) and fibroblast growth factor (FGF); and the fibroblast growth factor (FGF) is preferably fibroblast growth factor 4 (FGF-4).

Preferably, the sixth medium further comprises oncostatin M, DMSO, dexamethasone, and a first additive; and the first additive comprises ascorbic acid, BSA-FAF, hydrocortisone, transferrin, human insulin, gene recombinant human epidermal growth factor and GA-1000.

Preferably, a final concentration of HGF in the sixth medium is 10-30 ng/mL.

Preferably, a final concentration of FGF-4 in the sixth medium is 10-30 ng/mL.

Preferably, a final concentration of the oncostatin M in the sixth medium is 30-70 ng/mL.

Preferably, a final concentration of DMSO in the sixth medium is 0.1-1 w/w %.

Preferably, a final concentration of B27 in the sixth medium is 1-3 w/w %.

Preferably, a final concentration of the dexamethasone in the sixth medium is 80-120 nM.

More preferably, the first additive consists of seven components in a Single Quots kit: 0.5 mL of ascorbic acid, 5 mL of Bovine Serum Albumin, Fatty Acid Free (BSA-FAF), 0.5 mL of hydrocortisone, 0.5 mL of transferrin, 0.5 mL of human insulin, 0.5 mL of recombinant human epidermal growth factor and 0.5 mL of GA-1000, and the Single Quots kit is purchased from Lonza, with Cat. No. of CC-4182.

The basal culture medium of the sixth medium comprises a hepatocyte basal medium.

The sixth medium is used to induce hepatoblast organoids to differentiate into mature hepatocyte organoids.

Preferably, the medium composition includes a fourth medium, and the fourth medium comprises growth factors and BMP signaling pathway activators.

Preferably, the growth factors in the fourth medium comprise at least one of epidermal growth factor (EGF), platelet-derived growth factor (PDGF), fibroblast growth factor (FGF), hepatocyte growth factor (HGF), insulin-like growth factor-I (IGF-1), IGF-II, leukaemia inhibitory factor (LIF), nerve growth factor (NGF), oncostatin M (OSM), platelet-derived endothelial cell growth factor (PDECGF), transforming growth factor-a (TGF-α) and vascular endothelial cell growth factor (VEGF).

More preferably, the growth factors in the fourth medium comprise hepatocyte growth factor (HGF) and fibroblast growth factor (FGF); and the fibroblast growth factor (FGF) is preferably fibroblast growth factor 4 (FGF-4).

Preferably, a final concentration of HGF in the fourth medium is 10-30 ng/mL.

Preferably, a final concentration of FGF-4 in the fourth medium is 10-30 ng/mL.

Preferably, the BMP signaling pathway activators in the fourth medium comprise at least one of BMP2, BMP4, SB4, SJ000291942, SJ000063181, SJ000370178, isoliquiritigenin, diosmetin, apigenin and biochanin.

More preferably, the BMP signaling pathway activators in the fourth medium comprise BMP4 and BMP2.

Preferably, a final concentration of BMP4 in the fourth medium is 5-15 ng/mL.

Preferably, a final concentration of BMP2 in the fourth medium is 5-15 ng/mL.

Preferably, the fourth medium also comprises FBS, glutamax, human insulin, 1-thioglycerol, DMSO and dexamethasone.

Preferably, a final concentration of FBS in the fourth medium is 10-30 w/w %.

Preferably, a final concentration of glutamax in the fourth medium is 0.5-1.5 w/w %.

Preferably, a final concentration of human insulin in the fourth medium is 0.05-0.252 U/mL.

Preferably, a final concentration of 1-thioglycerol in the fourth medium is 0.2-0.4 mM.

Preferably, a final concentration of DMSO in the fourth medium is 0.4-0.6 w/w %.

Preferably, a final concentration of dexamethasone in the fourth medium is 50-150 nM.

Preferably, basal medium in the fourth medium is at least one of RPMI1640 medium and IMDM medium.

The fourth medium is used to induce the differentiation of endoderm cells into hepatoblasts.

Preferably, the medium composition includes a first medium, and the first medium comprises Activin A and a GSK-3 inhibitor.

Preferably, the GSK-3 inhibitor comprises at least one of B216763, TWS119, NP031112, SB216763, CHIR-98014, AZD2858, AZD1080, SB415286, LY2090314, and CHIR-99021.

More preferably, the GSK-3 inhibitor in the first medium comprises CHIR-99021.

Preferably, a final concentration of the Activin A in the first medium is 60-100 ng/ml.

Preferably, the final concentration of the Activin A in the first medium is 70-90 ng/ml.

Preferably, a final concentration of the GSK-3 inhibitor in the first medium is 1-5 M.

Preferably, basal medium of the first medium is at least one of DMEM with high glucose, DMEM-F12 medium or RPMI1640 medium.

Preferably, the medium composition includes a second medium, and the second medium comprises Activin A and a KnockOut serum replacement.

Preferably, the final concentration of the Activin A in the second medium is 60-100 ng/ml.

Preferably, the final concentration of the Activin A in the second medium is 70-90 ng/ml.

Preferably, a final concentration of the KnockOut serum replacement in the second medium is 0.5-1.1 w/w %.

Preferably, basal medium of the second medium is at least one of DMEM with high glucose, DMEM-F12 medium and RPMIl640 medium.

Preferably, the medium composition includes a third medium, and the third medium comprises Activin A and a serum replacement.

Preferably, the final concentration of the Activin A in the third medium is 60-100 ng/ml.

Preferably, the final concentration of the Activin A in the third medium is 70-90 ng/ml.

Preferably, the final concentration of the serum replacement in the third medium is 5-11 w/w %.

Preferably, basal medium of the third medium is at least one of DMEM with high glucose, DMEM-F12 medium and RPMI1640 medium.

Preferably, the first medium, the second medium and the third medium are used to induce human pluripotent stem cells to differentiate into endoderm cells.

According to the second aspect of the present disclosure, a kit is provided, in which the kit includes at least one of the first medium, the second medium, the third medium, the fourth medium, the fifth medium and the sixth medium in the first aspect of the present disclosure.

Preferably, the kit includes the first medium, the second medium and the third medium in the first aspect of the present disclosure, and is used to induce the differentiation of human pluripotent stem cells into endoderm cells under 3D suspension conditions.

Preferably, the kit includes the fourth medium in the first aspect of the present disclosure, and is used to induce the differentiation of endodermal cells into hepatoblasts under 3D suspension conditions.

Preferably, the kit includes the fifth medium in the first aspect of the present disclosure, and is used to induce the differentiation of hepatoblasts into hepatoblast organoids and to maintain sustainable expansion of hepatoblast organoids under 3D suspension conditions.

Preferably, the kit includes the sixth medium in the first aspect of the present disclosure, and is used to induce the differentiation of hepatoblast organoids into hepatocyte organoids under 3D suspension conditions.

Preferably, the kit includes the first medium, the second medium, the third medium, the fourth medium, the fifth medium and the sixth medium in the first aspect of the present disclosure, and is used to induce the differentiation of stem cells into hepatocyte organoids under 3D suspension conditions.

According to the third aspect of the present disclosure, uses of the medium composition in the first aspect of the present disclosure or the kit in the second aspect of the present disclosure is provided:
(1) preparation of endoderm cells under 3D suspension conditions;
(2) preparation of hepatoblasts under 3D suspension conditions;
(3) preparation of hepatocytes under 3D suspension conditions;
(4) development and/or continuous expansion of hepatoblast organoids under 3D suspension conditions;
(5) development of mature hepatocyte organoids under 3D suspension conditions;
(6) preparation of products of endoderm cells which are differentiated from induce stem cells under 3D suspension conditions;
(7) preparation of products of hepatoblasts which are differentiated from induce stem cells under 3D suspension conditions;
(8) preparation of products of hepatocytes which are differentiated from induce stem cells under 3D suspension conditions;
(9) preparation of products for developing and/or continuously expandable hepatoblast organoids under 3D suspension conditions; and
(10) preparation of products for developing mature hepatocyte organoids under 3D suspension conditions.

Preferably, the expansion includes 3D suspension expansion.

Preferably, the stem cells are human-derived stem cells with multidirectional differentiation potential;

Preferably, the human-derived stem cells with multidirectional differentiation potential are human embryonic stem cells, human parthenogenetic stem cells, human induced pluripotent stem cells, mesenchymal stem cells, adipose-derived stem cells or cord blood stem cells; Preferably, the stem cells are stem cell aggregates.

Preferably, the preparation method of the stem cell aggregates includes the following steps: mixing the stem cells with digestive solution and incubating, discarding the digestive solution and resuspending in mTeSR1 medium containing Rock inhibitor, and inoculating into fresh mTeSR1 culture medium containing the Rock inhibitor at a density of 300,000 to 1,000,000/well, replacing the culture medium with the mTeSR1 medium next day and culturing for additional 2-6 days.

Preferably, the Rock inhibitor is Y-27632.

Preferably, a final concentration of the Rock inhibitor in the mTeSR1 medium is 8-12 PM.

The function of Rock inhibitor used in the present disclosure is to maintain the vitality and survival ability of cells at cell inoculation during cell passage. However, it has been shown that Y-27632 only maintains cell viability at day 0 during the expansion of HB organoids, but it does not help much in the subsequent proliferation of HB organoids, thus, Y-27632 is not included in the expansion of the HB organoids.

According to the fourth aspect of the present disclosure, a method for developing and/or continuously expandable hepatoblast organoids is provided, including the steps of expanding and culturing hepatoblast organoids using the fifth medium in the first aspect of the present disclosure.

Preferably, in order to further improve the efficiency of the cell survival at the inoculation, 8-12 μM of Rock inhibitor can be added when hepatoblasts are inoculated.

Preferably, the Rock inhibitor is Y-27632.

Preferably, the expansion culture specifically comprises to passage hepatoblasts to obtain hepatoblast organoids, and to sustainably expand hepatoblast organoids under 3D suspension conditions using the fifth medium in the first aspect of the present disclosure.

Preferably, a passage ratio is 1: (3-5).

Preferably, a passage cycle is 4-8 days.

Preferably, the hepatoblasts can be hepatoblasts which are differentiated from endoderm cells under 3D suspension conditions using the fourth medium according to the first aspect of the present disclosure.

Preferably, the culture time for inducing endodermal cells to be differentiated into hepatoblasts is 100-188 hours.

Preferably, the culture time for inducing endodermal cells to be differentiated into hepatoblasts is 120-168 hours.

Preferably, the endoderm cells can be endoderm cells derived from the differentiation of stem cells induced by the first medium, the second medium, and the third medium under 3D suspension conditions in the first aspect of the present disclosure.

Preferably, the culture time of the first medium is 18-30 hours.

Preferably, the culture time of the second medium is 18-30 hours.

Preferably, the culture time of the third medium is 18-30 hours.

According to the fifth aspect of the present disclosure, a method for developing hepatocyte organoids is provided, including the steps of culturing hepatoblast organoids using the sixth medium under 3D suspension conditions in the first aspect of the present disclosure.

Preferably, the method specifically comprises to induce hepatoblast organoids to be differentiated into mature hepatocyte organoids under 3D suspension conditions using the sixth medium in the first aspect of the present disclosure.

Preferably, the culture time of the sixth medium is 120-240 hours.

Preferably, the culture time of the sixth medium is 120-192 hours.

Preferably, the culture time of the sixth medium is 130-168 hours.

Preferably, the hepatoblast organoids are induced to hepatocyte organoids at a density of 1: (1-4).

Preferably, the hepatoblast organoids are hepatoblast organoids developed under 3D suspension conditions or continuously expandable hepatoblast organoids under 3D suspension conditions by the method in the fourth aspect of the present disclosure.

According to the sixth aspect of the present disclosure, uses of hepatoblast organoids developed by the method in the fourth aspect of the present disclosure or hepatocyte organoids developed by the method in the fifth aspect of the present disclosure is provided in any one of the following:
1) drug screening, development and/or toxicity analysis related to liver diseases;
2) serving as hepatocyte source for bioartificial liver devices;
3) studying the mechanisms on the pathogenesis of liver diseases;
4) preparing products for treating liver diseases;
5) developing the modeling of liver diseases;
6) testing hepatotoxicity; and
7) liver tissue engineering and regenerative medicine.

The beneficial effects of the present disclosure are as follows:

The present disclosure provides a kit for inducing the differentiation of stem cells into hepatoblast organoids and/or hepatocyte organoids. Cytokines and the amount thereof are reduced in the kit, and the Matrigel at low concentration is used to differentiate and culture the organoids, which more efficiently supports the long-term maintenance and expansion of hepatoblast organoids and/or hepatocyte organoids. In the presence of Matrigel, hepatocytes in mature hepatocyte organoids are in polarized state that is similar to in vivo liver tissues, and are significantly different from non-polarized hepatic spheroids under 3D suspension culture conditions in terms of structure.

The present disclosure further provides a method for culturing and differentiating human pluripotent stem cells into hepatoblast (HB) organoids that can be passaged and expanded for a long time under 3D suspension culture conditions and inducing HB organoids to become mature hepatocyte organoids under 3D suspension culture conditions. First, a method is established for culturing hPSCs under 3D culture conditions and efficiently inducing hPSCs to differentiate into hepatoblasts under 3D suspension culture conditions. Furthermore, the culture system is optimized to enable hepatoblasts to grow into hepatoblast organoids that can be stably passaged, expanded and cryopreserved for a long term under 3D suspension culture conditions. Finally, the long-term expanded hepatoblast organoids are induced to differentiate into functional mature hepatocyte organoids under 3D suspension culture conditions. Since the entire processes under 3D suspension culture conditions is from culturing hPSCs and efficiently inducing the differentiation of hPSCs into hepatoblasts, to sustainably expanding hepatoblast organoids, finally to efficiently differentiating expanded hepatoblast organoids into functional hepatocyte organoids, thus the integration of hPSC culture and expansion, and the differentiation and long-term expansion of hepatoblast organoids as well as the productions of functional hepatocyte organoids, is eventually achieved at manufacturing purpose.

Moreover, the culture method of the present disclosure can better simulate the in vivo microenvironment. Compared with hepatocytes obtained under 2D culture conditions, hepatocytes produced under this 3D culture conditions are more mature and have stronger function. Low concentration of Matrigel (5%) is used during the entire culture process, which enables large-scale expansion and greatly reduces the cost. Finally, hepatoblast organoids that can be continuously expanded, passaged and cryopreserved under 3D suspension culture conditions have been developed (to date it has been expanded for 20 passages and can be continuously passaged), and this technology can achieve culture and expansion of organoids in vitro at large-scale and is expected to solve the bottleneck on producing functional hepatocytes at large-scale in the industry. The present disclosure utilizes a long-term in vitro maintenance method of hepatoblast organoids derived from human pluripotent stem cells to consume less medium and cytokines and shorten the differentiation time. The number of cells from hepatoblast organoids can be expanded from 3×10⁶ to the total of 10¹² within one month, which meets the needs on functional hepatocytes in clinical treatments, drug screening and development, drug toxicology analysis and other fields at aspects of cell yield, function, costs.

### BRIEF DESCRIPTION OF DRAWINGS

FIG. 1 is the illustration for the differentiation of human pluripotent stem cells into hepatoblasts, the induction and expansion of hepatoblast organoids from hepatoblasts, and the differentiation of mature hepatocyte organoids from hepatoblast organoids; FIG. 1A represents the entire process of the differentiation; FIG. 1B is images of cell spheroids at various stages during the differentiation, with a scale bar=100 μm.

FIG. 2A-2B are the examinations of hPSC aggregates during the expansion stage; FIG. 2A is an image of hPSC aggregates at day 5, with a scale bar=100 μm; and FIG. 2B is the expression levels of hPSC pluripotent markers OCT4, NANOG, TRA-1-60 and SSEA4 detected by immunofluorescence staining, with a scale bar=100 μm.

FIG. 3A-3D are the examinations for the differentiation at DE stage; FIG. 3A is an image of DE cell spheroids at day 3, with a scale bar=100 μm; FIG. 3B is the expression levels of endoderm-related genes in DE, as detected by qPCR FIG. 3C is the expression levels of endoderm markers CXCR4 and SOX17, as detected by flow cytometry; and FIG. 3D is the expression level of endoderm marker FOXA2, as detected by immunofluorescence staining, with a scale bar=100 μm.

FIG. 4A-4D are the examinations for the differentiation at hepatoblast stage; FIG. 4A is an image of hepatoblast spheroids at day 6, with a scale bar=100 μm; FIG. 4B is the expression levels of hepatoblast-related genes, as detected by qPCR; FIG. 4C is the expression levels of hepatoblast markers AFP and EPCAM in hepatoblast spheres compared to those in H9 cells, as detected by flow cytometry; and FIG. 4D is the expression levels of hepatoblast markers EPCAM, HNF4A and AFP, as detected by immunofluorescence staining, with a scale bar=100 μm.

FIG. 5A-5H are the examinations of HB organoids after long-term culture; FIG. 5A is an image of HB organoids at passage 10, with a scale bar=100 μm; FIG. 5B is an image of HE-stained section of HB organoids at passage10, with a scale bar=100 μm; FIG. 5C is the doubling time of HB organoids at passages 5 and 15 respectively; FIG. 5D is the expansion curve of HB organoids during the long-term culture; FIG. 5E is the expression levels of HB organoid-related genes at passages 5 and 15 compared to those at passage 0 as control, as detected by qPCR; FIG. 5F is the expression levels of HB organoid-related markers AFP, ALB, EPCAM, Ki67 and TBX3, as detected by flow cytometry; FIG. 5G is the karyotype analysis of HB organoids at passage15 after long-term culture; FIG. 5H is the expression levels of HB organoid markers AFP, ALB, EPCAM, SOX9, Ki67, E-cad, CK19 and HNF4A, as detected by immunofluorescence staining, with a scale bar=100 μm.

FIG. 6A-6B are the examinations for the recovery of HB organoids after the cryopreservation at passage 10; FIG. 6A is the images of HB organoids cryopreserved at passage10 after the recovery at days 3 and 6, with a scale bar=100 μm; FIG. 6B is the expression levels of HB organoid-related markers AFP, ALB, EPCAM, Ki67 and TBX3, as detected by flow cytometry after the recovery of HB organoids cryopreserved at passage10.

FIG. 7A-7H are the expansion of HB organoids at large-scale in a stirred bioreactor; FIG. 7A is a schematic diagram of large-scale culture of HB organoids; FIG. 7B is images of HB organoids cultured in the bioreactor, with a scale bar=100 μm; FIG. 7C is the comparison of the number of HB organoids between in static suspension culture and in dynamic suspension culture; FIG. 7D is the comparison of the expansion capabilities of HB organoids between in static suspension culture and in dynamic suspension culture; FIG. 7E is the comparison of the doubling time of HB organoids between in static suspension culture and in dynamic suspension culture; FIG. 7F is the average diameters of HB organoids during the dynamic suspension culture in the bioreactors; FIG. 7G is the comparison of the expansion capabilities of HB organoids derived from different cell lines; FIG. 7H is the total cell number of HB organoids with long-term expansion in the bioreactors in one month.

FIG. 8A-8D are the examinations of mature hepatocyte organoids induced from HB organoids after long-term culture; FIG. 8A is images of mature hepatocyte organoids induced from HB organoids, with a scale bar=100 μm; FIG. 8B is the expression levels of mature hepatocyte-related genes in mature hepatocyte organoids compared to those in primary human hepatocytes (PHH), HB organoids and H9 cells, as detected by qPCR; FIG. 8C is the expression levels of mature hepatocyte-related markers ALB, A1AT and ASGPR in mature hepatocyte organoids, as detected by flow cytometry; and FIG. 8D is the expression levels of mature hepatocyte markers ALB, E-cad, ZO1, CK18, ASGPR, A1AT and HNF4A detected by immunofluorescence staining, with a scale bar=100 μm.

FIG. 9A-9I are the examinations of mature hepatocyte organoids induced from HB organoids after long-term culture; FIG. 9A is the secretion levels of ALB, as detected by ELISA; FIG. 9B is an image of hepatocyte organoids stained by PAS, with a scale bar=100 μm; FIG. 9C is the images of hepatocyte organoids one hour after the treatment with ICG or one hour after the removal of ICG, with a scale bar=100 μm; FIG. 9D is the expression levels of urea cycle-related genes in polarized hepatocyte organoids compared to primary human hepatocytes (PHH), HB organoids and polarized hepatocyte spheres, as detected by qPCR; FIG. 9E is the detection of urea synthesis ability in polarized hepatocyte organoids compared to primary human hepatocytes (PHH), HB organoids and polarized hepatocyte spheres; FIG. 9F is the expression levels of phase I metabolic enzyme-related genes after the inductions with or without rifampicin or omeprazole compared to those in hepatocyte organoids without the inductions by the inducers, primary human hepatocytes (PHH) and H9 cells, as detected by qPCR; FIG. 9G is the detections of metabolic function levels of phase I metabolic enzymes after the inductions with or without rifampicin or omeprazole in hepatocyte organoids after the inductions by the inducers rifampicin or omeprazole compared to those in hepatocyte organoids without the inductions by the inducers, as detected by the kit; FIG. 9H is the detections of hepatocyte polarized markers in polarized hepatocyte organoids, as detected by immunofluorescent staining; FIG. 9I is the detections of bile canaliculi in polarized mature hepatocyte organoids, as detected by CDFDA staining.

FIG. 10A-10I are the development of non-alcoholic fatty liver disease (NAFLD) model; FIG. 10A is the formation of lipid droplets after the induction with oil red (OA) with a scale bar=100 μm; FIG. 10B is the detections of triglyceride levels after the induction with OA; FIG. 10C is the detections of intracellular oxidative stress levels after the induction with OA; FIG. 10D is the quantitative statistics of oxidative stress levels; FIG. 10E is the expression levels of lipid accumulation-related genes after the induction with OA, as determined by qPCR; FIG. 10F is the detection for OA-induced lipid droplet levels after the treatments with spermidine, with a scale bar=100 μm; FIG. 10G is the quantitative statistics of intracellular lipid droplet levels; FIG. 10H is the detection of intracellular triglyceride levels; and FIG. 10I is the expression levels of lipid accumulation-related genes in OA-induced hepatocyte organoids after the treatment with spermidine, as determined by qPCR

FIG. 11A-11C are the development of the cellular model for drug screening/toxicity analysis; FIG. 11A is IC50 curves for drug screening; FIG. 11B is statistical values for IC50, as presented by bar chart; and FIG. 11C is statistical values for IC50, as presented by summary table.

FIG. 12 is the occurrence of the cholestasis in polarized hepatocyte organoids, as detected by CDFDA staining, with a scale bar=50 μm.

FIG. 13A-13G are the rescue of mice with acute liver failure by the treatment with polarized hepatocyte organoids; FIG. 13A is a schematic diagram of the development of model mouse with acute liver failure and the rescue of model mouse by the transplantation with polarized hepatocyte organoids; FIG. 13B is the statistics of mouse survival rates; FIG. 13C is the reduction of liver damage indicators ALT, AST, TBIL and ALP, and blood ammonia; FIG. 13D is the quantitative detection of human albumin secretion levels; FIG. 13E is the comparison on the appearance of mouse liver organs; FIG. 13F is the comparison on histology of mouse liver tissues, as detected by HE staining; FIG. 13G is the statistical liver damage areas between model mice.

FIG. 14A-14D are the effects of different Matrigel concentrations on the expansion of HB organoids; FIG. 14A is the morphologies of hepatoblast spheroids after removing Matrigel, with a scale bar=100 μm; FIG. 14B is the reduction of the expansion ability of hepatoblasts after removing Matrigel; FIG. 14C is the expression levels of hepatoblast-related markers after removing Matrigel, as determined by flow cytometry; FIG. 14D is the images of cells at day 5 after passaging without or with different concentrations of Matrigel, with a scale bar=100 μm.

FIG. 15A-15D are the screening of medium components for the expansion of HB organoids; FIG. 15A is the morphologies of cell spheroids in each group after removing single given growth factor or small molecule, with a scale bar=100 μm; FIG. 15B is the colony formation rates of cells in each group after removing single given growth factor or small molecule; FIG. 15C is the statistics for the proliferation marker Ki67 in each group after removing single given growth factor or small molecule, as determined by flow cytometry; FIG. 15D is the statistics for the expression levels of the proliferation marker Ki67 in each group after removing single given growth factor or small molecule.

### DETAILED DESCRIPTION

The concept and the technical effects produced in the present disclosure will be clearly and completely described below with reference to the embodiments, so as to fully understand the purpose, features and effects of the present disclosure. Obviously, the described embodiments are only some of the embodiments of the present disclosure, but not all. Based on the embodiments of the present disclosure, other embodiments obtained by those skilled in the art without exerting creative efforts are all included in the protective scope of the present disclosure.

### Definition

The term “stem cells” refer to cells that are capable of self-renewal and possess pluripotency or multipotency. Generally, stem cells can allow injured tissue to regenerate. Stem cells herein may be, but are not limited to, embryonic stem (ES) cells, induced pluripotent stem cells or tissue stem cells (also known as tissue-specific stem cells or somatic stem cells).

“Embryonic stem (ES) cells” are pluripotent stem cells derived from early embryos. Mouse ES cells were first established in 1981 and have also been used to generate knockout mice since 1989. In 1998, human ES cells were established, which are currently becoming available for regenerative medicine.

Unlike ES cells, tissue stem cells have limited differentiation potential. Tissue stem cells exist in specific locations in tissues of the organs and have an intracellular structure of undifferentiated state. Therefore, tissue stem cells often have low pluripotency. Tissue stem cells have a higher nuclear/cytoplasmic ratio and fewer intracellular organelles. Most tissue stem cells have low pluripotency, long cell cycles, and proliferation ability beyond the individual lifespan. Tissue stem cells are divided into several categories based on the locations from which the cells originate, such as skin system, digestive system, bone marrow system, nervous system, etc. Tissue stem cells in skin system include epidermal stem cells, hair follicle stem cells, etc. Tissue stem cells in digestive system include pancreatic (common) stem cells, hepatic stem cells, etc. Tissue stem cells in bone marrow system include hematopoietic stem cells, mesenchymal stem cells, etc. Tissue stem cells in nervous system include neural stem cells, retinal stem cells, etc.

“Induced pluripotent stem cells” (often abbreviated to iPS cells or iPSCs) refer to pluripotent stem cell types are artificially prepared from non-pluripotent cells (usually somatic cells) or terminally differentiated cells (e.g., fibroblasts, hematopoietic cells, muscle cells, neurons, epidermal cells, etc.) by introducing certain factors called reprogramming factors.

The term “differentiation” is the process by which relatively non-specialized cells form the offspring of at least one new specialized cell type.

The term “SB431542” also includes SB431542 and its salts, especially pharmaceutically acceptable salts.

The term “CHIR99021” includes CHIR99021 and its salts, especially pharmaceutically acceptable salts.

The term “Y-27632” also includes Y-27632 and its salts, especially pharmaceutically acceptable salts. The preferred pharmaceutically acceptable salt is Y-27632 2HCL.

The term “fibroblast growth factor (FGF)” refers to the polypeptide secreted by the pituitary gland and hypothalamus. It can promote the mitosis of fibroblast and the growth of mesodermal cells, also can stimulate the formation of blood vessels and plays roles in wound healing and limb regeneration. There are two types of FGF such as acidic (pI 5.6) and alkaline (pI 9.6), namely aFGF and bFGF.

The term “bone morphogenetic protein 4”, that is, BMP4, regulates the proliferation and differentiation of various cells during embryonic development.

### Example 1

A kit comprised the first medium, the second medium, the third medium, the fourth medium, the fifth medium and the sixth medium.

The first medium contains 1640 medium (Gibco, 61870036) supplemented with 80 ng/ml Activin A (Peprotech, 120-14) and 3 μM CHIR99021 (Selleck, CT99021);

The second medium contains 1640 medium supplemented with 80 ng/ml Activin A and 0.8% KSR (Thermo, 10828028);

The third medium contains 1640 medium supplemented with 80 ng/ml Activin A and 8% KSR

The fourth medium (HB medium) contains IMDM medium (Gibco, 31980030) supplemented with 20% FBS (Fetal Bovine Serum, VISTECH, SE100-B7953) and 1% w/w GlutaMAX (Gibco, 35050061), 0.126 U/mL human insulin (recombinant Human Insulin, Sigma, 91077C-1), 0.3 mM 1-thioglycerol (Sigma, M6145), 20 ng/mL FGF-4 (Peprotech, 100-18B), 20 ng/mL HGF (Peprotech, 100-39), 10 ng/mL BMP2 (Peprotech, 120-02), 10 ng/mL BMP4 (Peprotech, 120-05), 0.5% DMSO (dimethyl sulfoxide, MP Biomedical, 196055) and 100 nM dexamethasone (Sigma, D4902).

The fifth medium is hepatoblast (HB) organoid expansion medium supplemented with 5% Matrigel (Corning, 354230).

Hepatoblast (HB) organoid expansion medium contains IMDM medium (Gibco, 31980030) supplemented with 10% w/w FBS, 1% w/w ITS (biogems, 00-101), 1% w/w NEAA (Gibco, 11140050), 1% w/w GlutaMAX (Gibco, 35050061), 10 mM nicotinamide (Sigma, N0636), 5 μM CHIR99021, 10 μM SB431542 (Selleckchem, S1067), 10 μM FSK (MCE, HY-15371), 20 ng/mL FGF-4, 20 ng/mL BMP4 and 20 ng/mL EGF (Peprotech, AF-100-15).

The sixth medium is hepatocyte organoid maturation culture medium supplemented with 5% Matrigel (Corning, 354230).

Hepatocyte organoid maturation medium contains hepatocyte basal medium (Lonza, CC-3911) supplemented with Single Quots kit (Lonza, CC-4182), 20 ng/mL HGF, 50 ng/mL oncostatin M (Peprotech, 300-10), 0.5% DMSO, 100 nM dexamethasone (Sigma, D4902), 20 ng/mL FGF-4 and 2% w/w B27 (Gibco, 17504044).

### Example 2

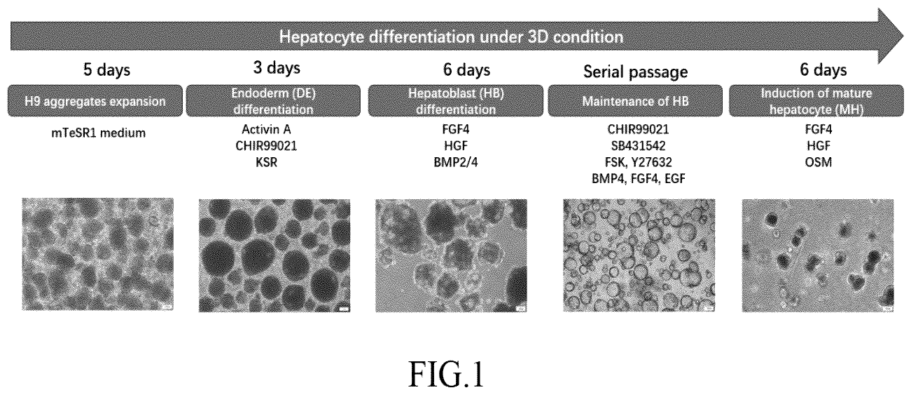

An efficient method for directed differentiation of hepatoblasts from human pluripotent stem cells (hPSCs) under 3D suspension culture condition, the illustration for the differentiation is shown in FIG. 1, which specifically includes the following steps:

1. Under 3D suspension culture conditions, human pluripotent stem cells were cultured and expanded in the form of aggregates as the following: cells were cultured in a cell culture incubator at 37° C. with 5% CO₂. Briefly, hPSC cells in good conditions (in which hPSC growth reached about 70%-80% confluence at days 4-5 after hPSC passaging, and the clone edges of hPSCs were regular without differentiated cells) cultured in 2D adherent conditions were selected to use for the expansion under 3D suspension conditions. After the medium was aspirated, 1 mL of PBS was added to wash the cells in the wells. After PBS was aspirated, 1 mL of GCDR (digestive enzyme) was added, and the cells was put back into CO₂ incubator to incubate for 4-6 minutes, then GCDR in the wells was aspirated. 1 mL of mTeSR1 medium containing 10 μM of Y-27632 was added to resuspend the cells, and the pipette was used to gently pipet up and down to obtain single cell suspension. Next single cell suspension was seeded into ultra-low adhesion 6-well plates at the density of 500,000 cells/mL in 3 mL per well. mTeSR1 medium was supplemented with 10 μM Y-27632 at day 1, and cells was cultured in the incubator. Next day the medium was replaced with regular mTeSR1 medium, and then the medium was changed every day. Thus, the culture and expansion of human pluripotent stem cells under 3D suspension culture conditions was completed to the fifth day.

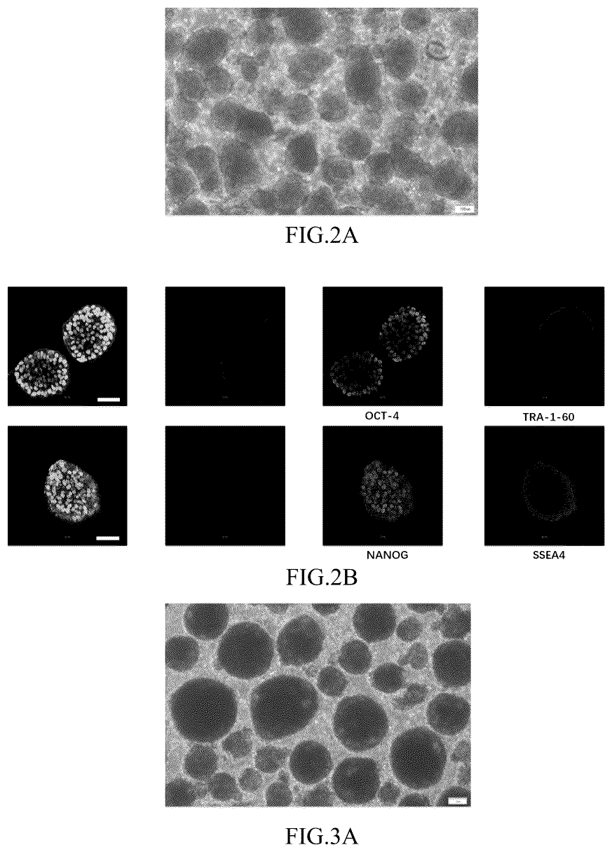

Part of cell spheroids were taken for quality examination, and the results are shown in FIG. 2A-2B. FIG. 2A is the morphology of hPSC aggregates under the light microscope. In addition, immunofluorescence staining was used to detect the expression of hPSC pluripotent markers at day 5 after the expansion under such condition. The results show that hPSC aggregates still highly express OCT4, NANOG, TRA-1-60 and SSEA4 (FIG. 2B), indicating that this culture condition can maintain the pluripotency of hPSCs and is suitable for subsequent differentiation.

2. After hPSC aggregates were collected, the aggregates were washed once with PBS, and then are used to start the differentiation into DE for 3 days as the following:

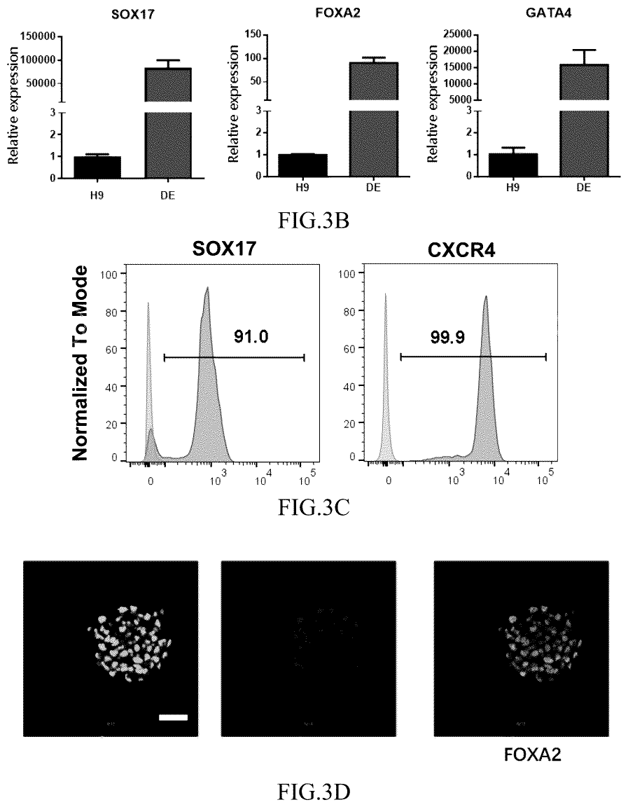

Under 3D suspension culture conditions, the aggregates of hPSCs were differentiated into definitive endoderm (DE): Briefly, the differentiation into DE requires changing different media every day. First of all, the medium for DE differentiation was prepared, the first medium was used at day 1, the second medium was used at day 2, and the third medium was used at day 3. 3 days after the differentiation, part of cell spheroids were taken for testing. The results are shown in FIG. 3A-3D. Compared with pluripotent stem cells in the undifferentiated state, after DE differentiation, three endoderm-related genes, SOX17, FOXA2 and GATA4, were highly expressed. Moreover, high expression levels of three endoderm markers, CXCR4, SOX17 and FOXA2, were verified by flow cytometry and immunofluorescence staining after the differentiation, indicating that higher purity of DE cells were obtained at this time, suitable for subsequent hepatocyte differentiation. 3. After being collected, DE cell spheroids were washed once with PBS, and then the fourth medium were used to start hepatoblast differentiation for 6 days. The medium was changed daily, and under 3D suspension culture conditions, DE cell spheroids can be differentiated into hepatoblast spheroids.

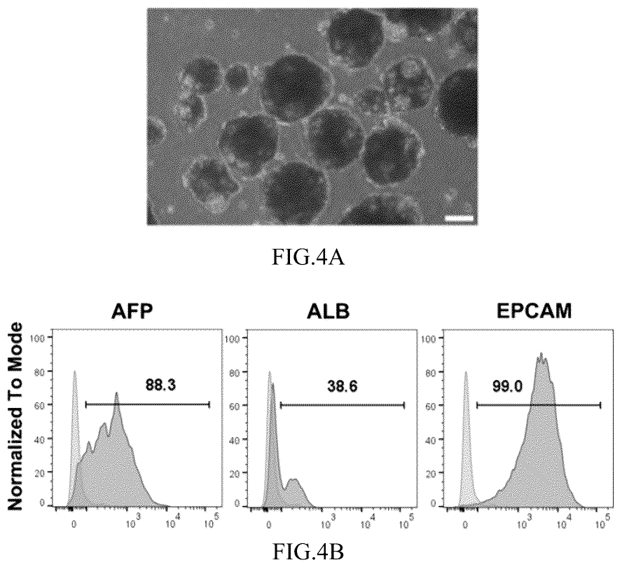

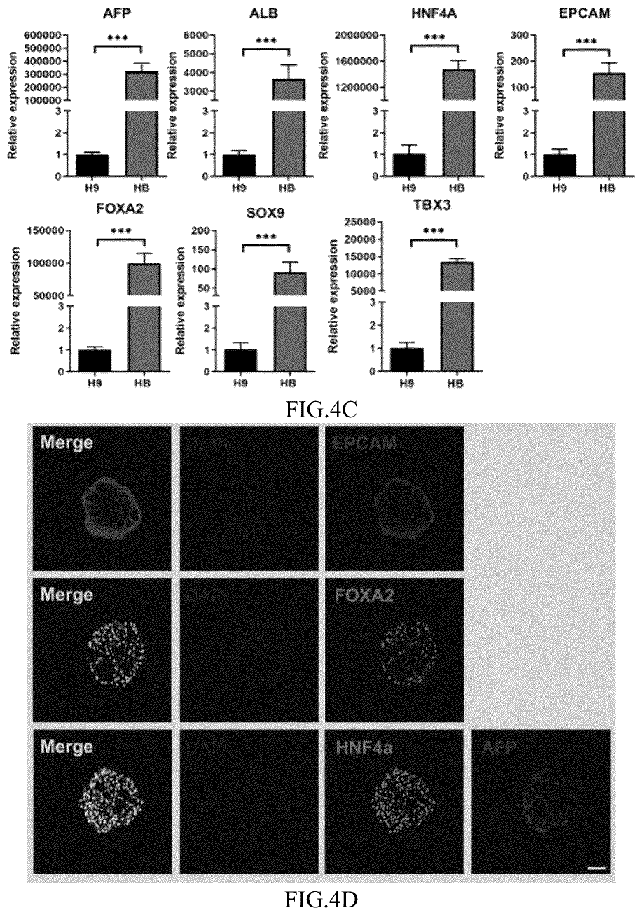

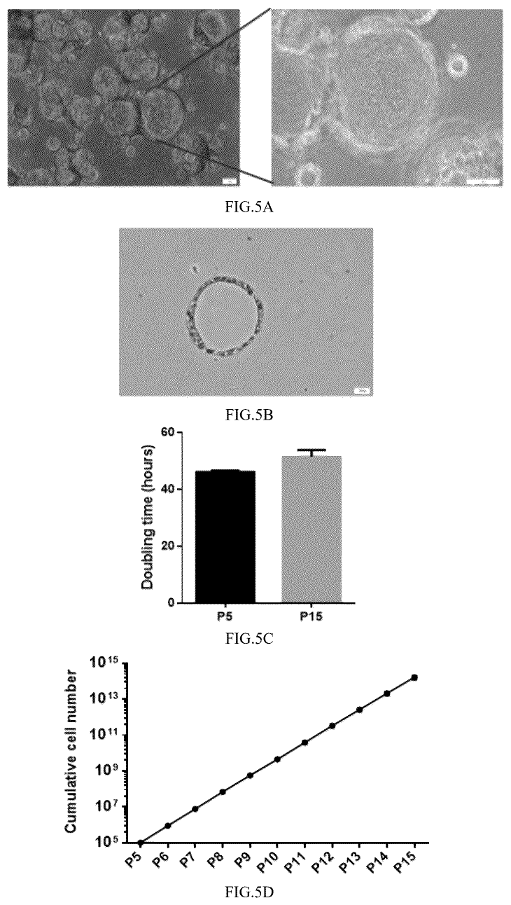

Six days after the differentiation, part of the cells were taken for testing. The results are shown in FIG. 4A-4D. Six days after the differentiation, cell spheroid structure was changed from a solid DE cell spheroid to a relatively hollow hepatoblast (HB) spheroid, which highly expressed alpha fetal protein (AFP) and EPCAM, indicating that most of cells were in hepatoblast state. The relative gene expression levels were analyzed by qPCR, and the results shows that genes related to hepatoblasts were highly expressed in this state, indicating gene expression characteristics of hepatoblasts. Finally, immunofluorescent staining was further used to verify high expression of hepatoblast markers, EPCAM, HNF4A and AFP, these results indicate that through the differentiation method in this example, high purity of hepatoblasts can be efficiently obtained for subsequent experiments.

### Example 3

An efficient method for long-term expansion and maintenance of hepatoblast (HB) organoids under 3D suspension culture conditions, the fifth medium was used for the induction and culture, specifically including the following steps:

1. Six days after the differentiated, HB spheroids were digested into single cells by TrypLE, with the following steps: Briefly, HB spheroids were collected, and washed once with PBS, then PBS was aspirated, after 1 mL of TrypLE was added, HB spheroids were put into the incubator for the digestion for 5-7 minutes, then gently pipetted up and down into single cell suspension. 500 μL of HB medium was added to terminate the digestion, then centrifugation was performed at 1,500 rpm for 5 minutes. After the supernatant was aspirated, single cell suspension was resuspended with HB medium, and then single cell suspension was seeded into new ultra-low adhesion 6-well plates at the density of 200,000/mL, each well was inoculated with 2.5 mL of HB organoid expansion medium. In addition, 10 μM of Y-27632 was required to add at the inoculation.

2. At the first day after the inoculation, the medium was not needed to be changed, 5% volume of Growth factor reduced Matrigel was directly added into culture medium. Matrigel was rich in extracellular matrix and could provide extracellular matrix to support hepatoblasts under 3D suspension culture conditions, thus the support of Matrigel was vital in this system. By adding 5% of Matrigel, hepatoblasts could form hepatocyte organoid-like vacuolated cell spheroid structure. In control group without adding Matrigel, hepatoblasts gradually died, and could not maintain the expansion for a long time.

3. At the second day, 1 mL of hepatoblast (HB) organoid expansion medium was supplemented, then next day 2 mL of medium was replaced every day. When the medium was replaced, the well plates were slightly tilted, so that cell spheroids could sink to the bottom of the well; and after the supernatants were aspirated, fresh medium was added.

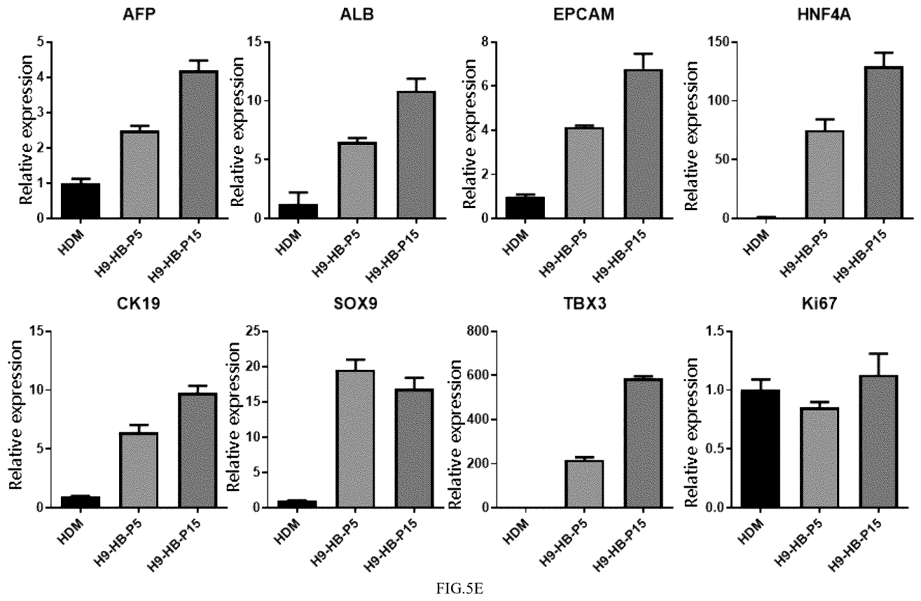

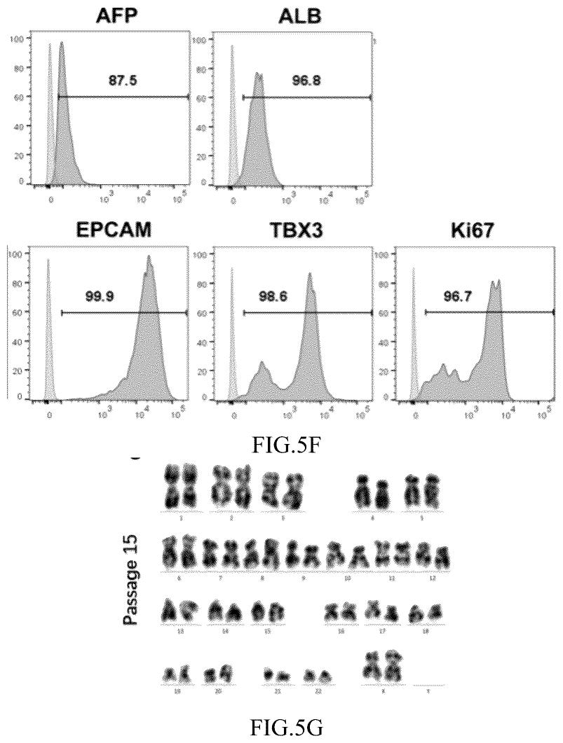

4. Cells were passaged 6-7 days after the culture. At this time, cell spheroids were presented as hepatocyte organoid-like hollow cell spheroids, as shown in FIG. 5A. Cell were passaged as the following: all cell spheroids were collected and centrifuged at 1,000 rpm for 2 minutes. After the supernatants were aspirated, cell spheroids were washed once with PBS and then centrifuged to aspirate PBS. 1 mL of TrypLE was added and put into the incubator to digest for 5-7 minutes, then gently pipetted up and down to produce single cell suspension. 500 μL of HB medium was added to terminate the digestion, and cells were centrifuged at 1,500 rpm for 5 minutes. After the supernatants were aspirated, single cell suspension was resuspended in 400 μL of HB medium when cells were passaged at a ratio of 1:4. Meanwhile, 400 μL of Matrigel was added, mixed gently and placed on ice for further use. 1.8 mL of HB expansion medium was added to each well of the 6-well cell culture plates, and then 200 μL of the mixture of cell suspension and Matrigel was added to each well. Next, 1 mL of pipette was used to gently pipet up and down to mix the culture system, then cells were placed back to the incubator for the culture. At the first day after passaging, the formation of a thin layer of Matrigel and small cell spheroids wrapped by Matrigel could be observed. Three to four days after the culture, cell spheroids entered the exponential growth phase and solid cell spheroids were changed to hepatocyte organoid-like hollow cell spheroid shape, and expanded quickly. Six to seven days after the expansion, the passaging should be performed again.

5. Cryopreservable feature with the capacity of the recovery: during long-term culture, HB organoids could be cryopreserved and recovered, and stored in liquid nitrogen for a long time. The survival rate of recovered primary hepatocytes after the cryopreservation is generally very low, and liver function is seriously lost after the recovery, while the recovery of HB organoids was very good after the cryopreservation. After the recovery, HB organoids could still form hollow organoids and maintain efficient expansion capabilities. The cryopreservation included the steps as the following: all organoids were collected, and centrifuged at 1,000 rpm for 2 minutes. After the supernatants were aspirated, HB organoids were washed once with PBS, then centrifuged to aspirate PBS. 1 mL of TrypLE was added, and cells were placed in the incubator to be digested for 5-7 minutes, and then gently pipetted up and down into small cell clusters. 500 μL of HB medium was added to terminate the digestion, and cells were centrifuged at 1,500 rpm for 5 minutes. After the supernatants were aspirated, 1 mL of mFreSR medium was added, cells were gently pipetted to mix the small cell clusters well, and transferred to the cryovial for the cryopreservation.

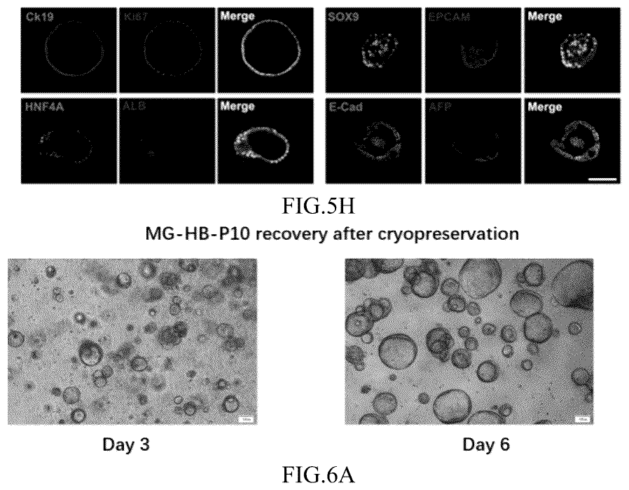

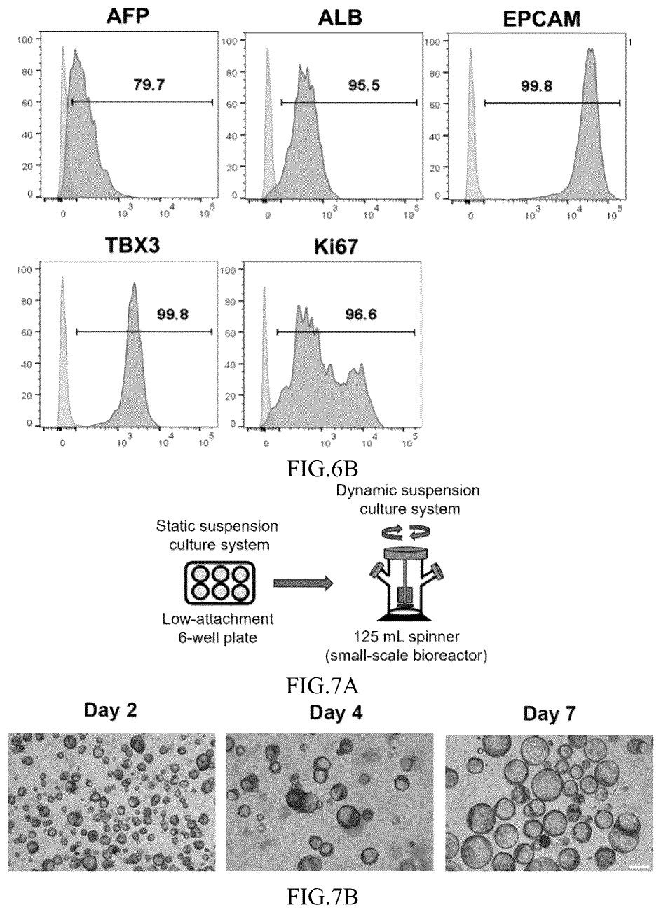

Part of HB organoids after long-term culture were taken to test, and results were as shown in FIG. 5A-5H and FIG. 6A-6B. First of all, it can be observed that the shapes of cell spheroids is obviously different from previous one. Cell spheroids at this stage were very close to the hollow cell spheroid-like structure of hepatocyte organoids, and HE staining results also confirm that cell spheroids at this stage was composed of only one layer of cells with the hollow sphere-like structure. Under this culture system, the doubling time of HB cells was about 45 hours, and they were passaged once every 6 days averagely. The cell number could be stably increased 6-8 times per passage. If the starting number of hepatoblasts was 100,000, 10¹¹ in total cells could be obtained through the expansion within 7 passages. After the long-term expansion, HB organoids still highly expressed genes related to hepatoblasts, as determined by qPCR. In addition, flow cytometry and immunofluorescent staining were also performed to verify high protein expression of hepatoblast markers such as AFP, ALB, HNF4A, EPCAM, SOX9, TBX3, TBX3, CK19 and TBX3 in HB organoids. Moreover, flow cytometry analysis also proved that HB organoids had a high positive rate of Ki67, indicating that cells were in active proliferation state. In addition, karyotype analysis was performed to determine the chromosome stability of HB organoids at passage 15 after long-term culture. The results show that the chromosomes are all normal and no occurrence of chromosomal mutations, indicating that the culture system of the present disclosure is very stable.

HB organoids were cryopreserved at passage 10, and thawed cells still could assemble cell spheroids after the recovery, and have high recovery efficiency and spheroid-forming efficiency. And newly-assembled cell spheroids were also able to form hepatocyte organoid-like structures to become HB organoids. Flow cytometry analysis shows that cells still maintain the expression of characteristic markers of hepatoblasts after the recovery, with high expression of AFP, ALB, EPCAM, TBX3 and Ki67, indicating that cells still have the recovery ability to maintain the characteristics of hepatoblasts after long-term cryopreservation.

### Example 4

An efficient method for the expansion of HB organoids at large-scale in a bioreactor (spinner flask). The specific steps included the followings:

HB organoids were collected after long-term culture and centrifuged at 1,000 rpm for 1 minutes, and washed once with PBS. After the supernatants were aspirated, 1 mL of TrypLE was added and placed at 37° C. for cell digestion. Five minutes after the digestion, serum-containing medium was added for the neutralization to terminate the digestion, then the organoids were pipetted up and down into single cell suspension and centrifuged at 1,500 rpm for 5 minutes. After the supernatants were aspirated, HB organoid expansion medium was used to resuspend cells, and then cell counting was performed. 3 million single cells were inoculated into low-attachment 10 cm culture dish for 24 hours to form small organoids under static suspension culture. In low-attachment 10 cm culture dish, 10 ml of medium with 5% Matrigel (500 μl) was used for the culture. Twenty-four hours after the culture, all small HB organoids in low-attachment 10 cm culture dish were gently transferred into spinner flasks. The medium was supplemented to 30 ml, and 1 ml of Matrigel was used to supplement to 5% in the total system. The speed of the rotation was set to 60 rpm with continuous stir. During the culture in stirred bioreactors, the medium was changed half a daily. During the medium change, spinner flask was left to rest for 5 min first, so that organoids were allowed to settle to the bottom of the flasks, and then the supernatants were aspirated and fresh medium was supplemented. Cells were harvested 7 days after the culture.

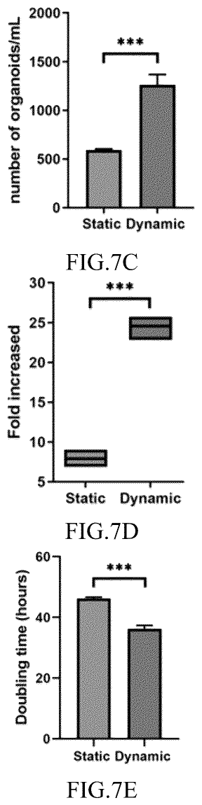

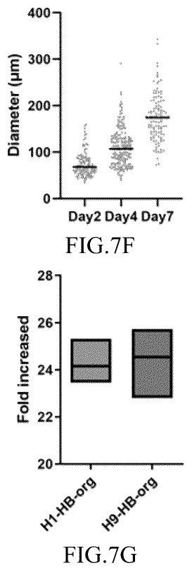

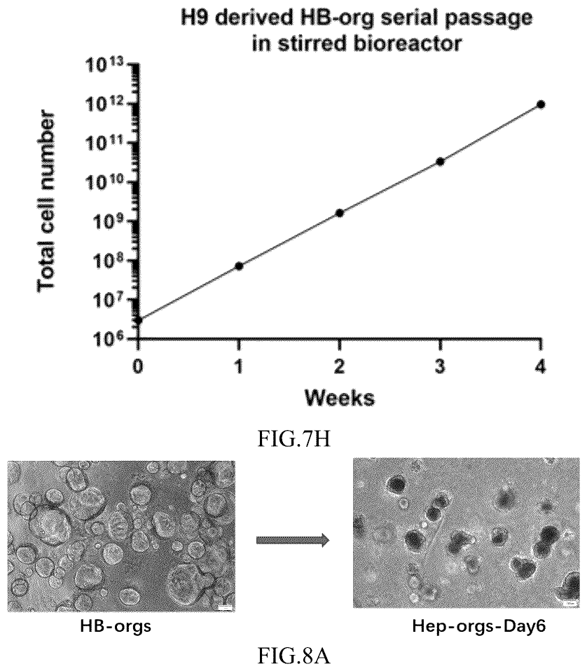

The results show that HB organoids expanded in the bioreactors are still in a typical hollow cell organoids as ones cultured in 6-well cell culture plates. Compared to static suspension culture, dynamic suspension culture can yield twice as many organoids as static suspension culture does in number, and the efficiency of the expansion in the bioreactors are similar, indicating the stability of this expansion system. Finally, HB organoids differentiated from H9 cells could be cultured and expanded in the bioreactors for a month, which was consistent with long-term culture and expansion in cell culture plates/dishes also can be stably and continuously be expanded in the bioreactors. More importantly, the average cell number can be increased about 24 time per passage. Thus, a total number of cells could reach around 10¹² by the accumulation of the expansion after consecutive 4 passages, this yield of cells could meet the clinical demand on hepatocyte transplantation (FIG. 7A-7H).

### Example 5

An efficient method for inducing the differentiation of long-term cultured HB organoids into mature hepatocyte organoids and uses.

1. Long-term cultured HB organoids were collected and centrifuged at 1000 rpm for 1 minutes, and washed once with PBS. After the supernatants were aspirated, the prepared sixth medium was added to gently resuspend HB organoids. Then HB organoids were transferred to new ultra-low attachment 6-well cell culture plates for the culture. During transferring, 5% Matrigel was supplemented to the medium to support the growth of mature hepatocyte organoids. Briefly, when HB organoids were transferred to a well in the six-well cell culture plate, firstly 1.9 mL of sixth medium was used to gently resuspend HB organoids, and 0.1 mL of Matrigel pre-chilled on ice was added into the medium to mix well gently, then 2 mL of the mixed suspension was transferred to the wells for the culture. The medium was changed every two days. At this time, only fresh hepatocyte organoid maturation medium was used to replace old one without the need for additional Matrigel. One well of HB organoids can be induced to differentiate towards mature hepatocyte organoids at a density of at least 1:2.

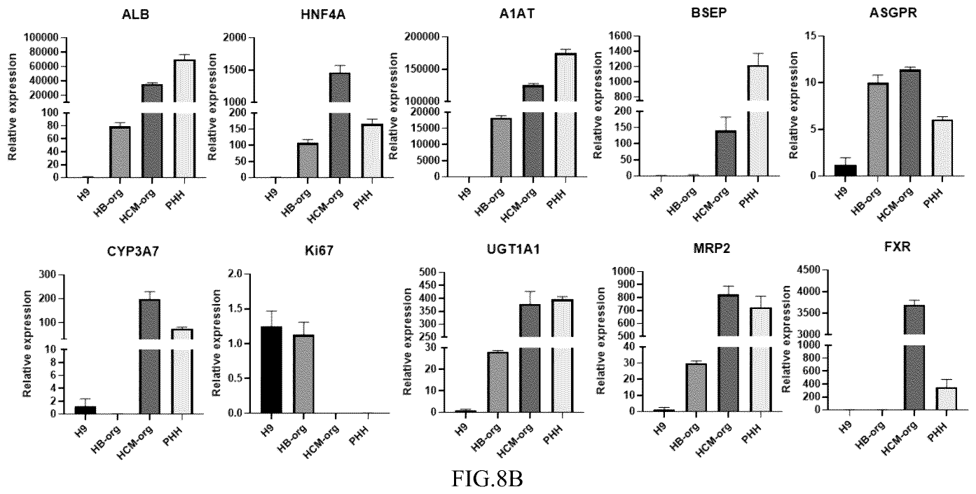

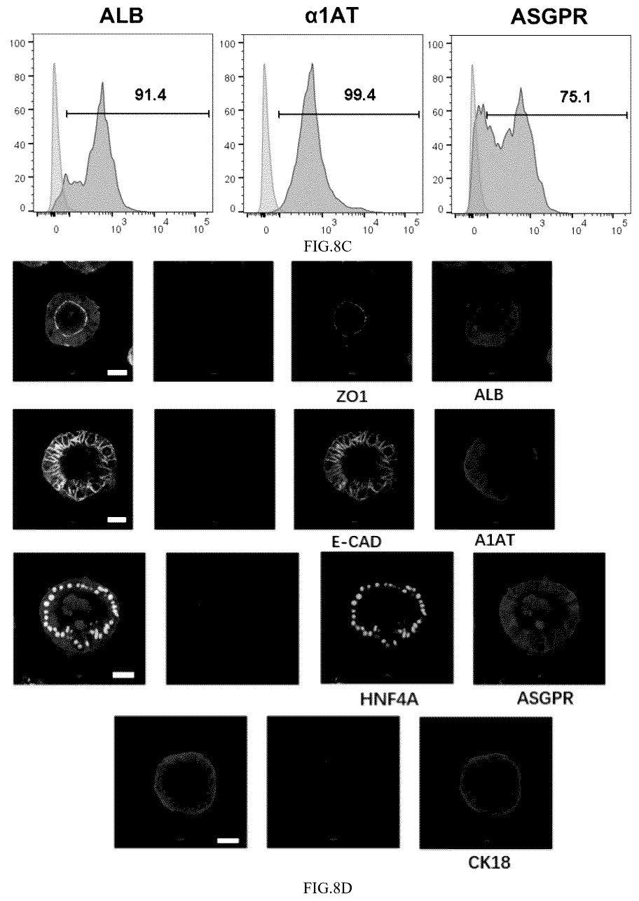

Six days after the differentiation, part of hepatocyte organoids were taken for detecting the maturity of liver function, and the results were shown in FIG. 8A-8D. Mature hepatocyte organoids highly expressed liver function characteristic genes ALB, A1AT, HFN4A, ASGPR, FXR and UGTIA1 at this stage, as determined by qPCR analysis. The expression levels of most genes were close to or even higher than those of freshly isolated primary human hepatocytes (PHH) cultured for 24 hours in vitro. In addition, the expression of proliferation gene Ki67 also was changed from high level in HB organoids to low level in hepatocyte organoids after the maturation, which was similar to those in PHH. Flow cytometry and immunofluorescent staining analysis were further performed to verify the high expression of mature hepatocyte-specific protein markers ALB, A1AT, ASGPR, ZO1, CK18, HNF4A and E-cad in hepatocyte organoids after the induction to the maturation, confirming that cells at this stage were indeed mature hepatocytes.

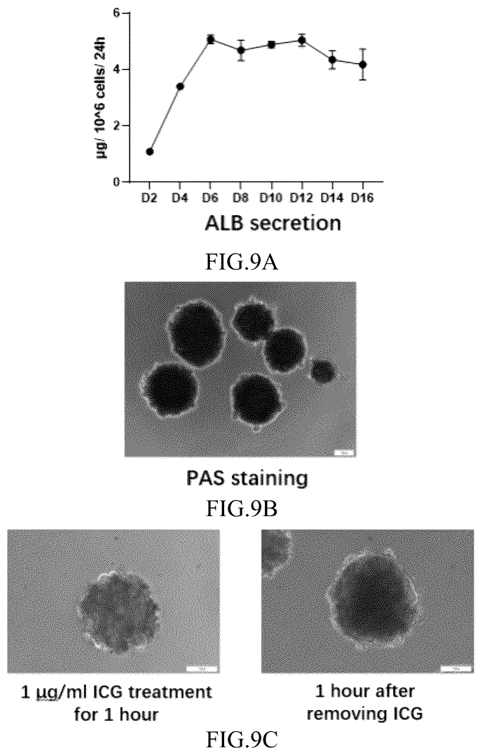

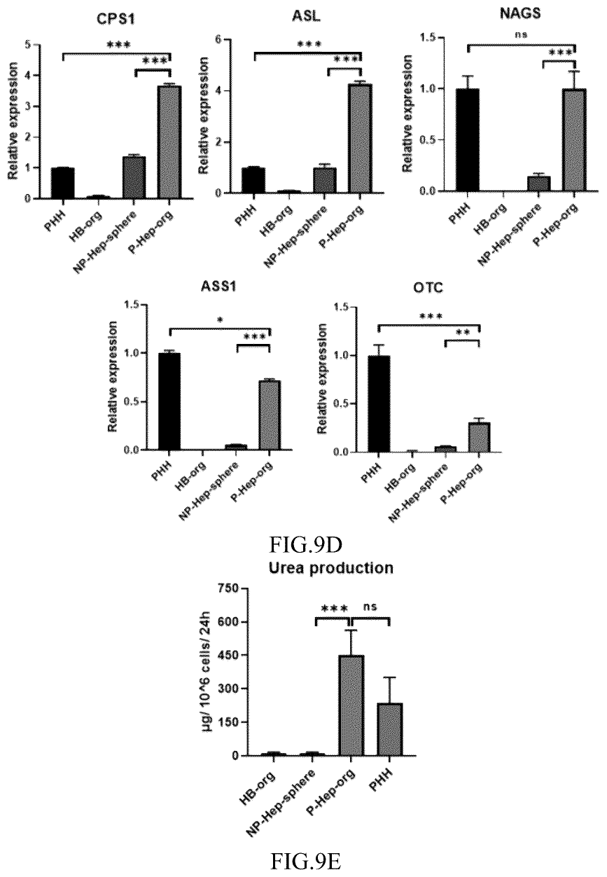

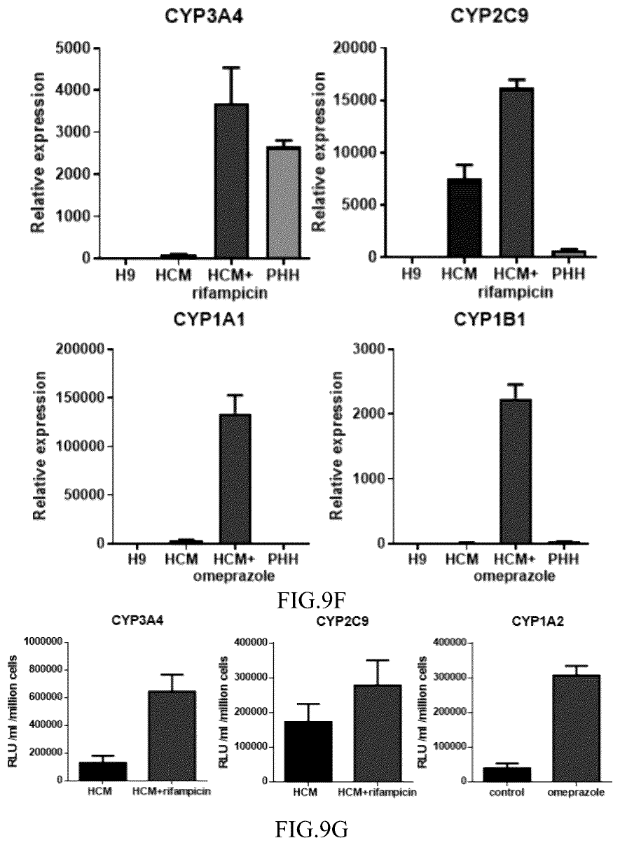

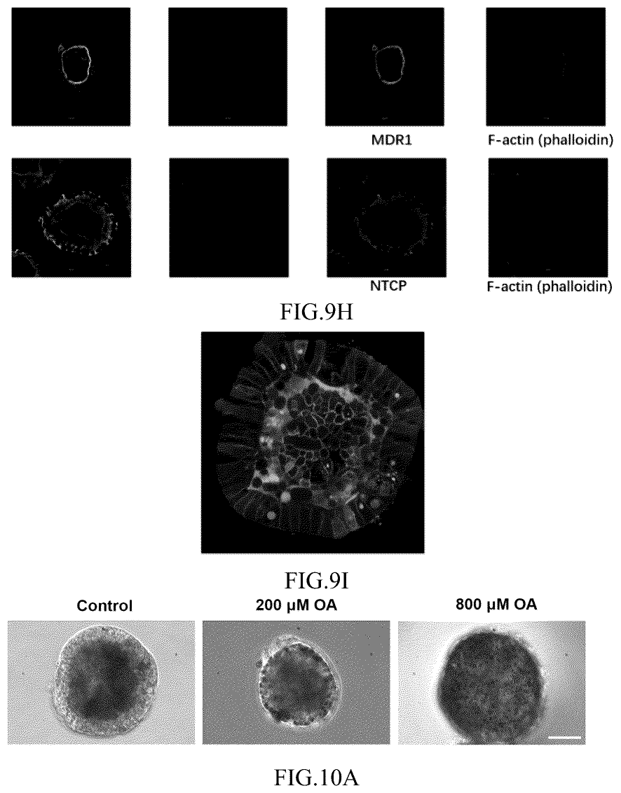

A series of tests for in vitro liver function were then performed, as shown in FIG. 9A-9I. ALB secretion level could reach 5 μg/mL in culture medium. The result of glycogen staining using PAS demonstrates that hepatocyte organoids have the capacity to store glycogens. Moreover, indocyanine green (ICG) was used for staining hepatocyte organoids to determine its capacity of cellular uptake and excretion of ICG. One hour after staining, hepatocyte organoids turned green, and 1 hour after the removal of ICG in the culture medium by washing off, hepatocyte organoids excreted green ICG, indicating that hepatocyte organoids have transport ability. By testing the urea metabolism ability, it had been shown that ammonia metabolism-related genes CPS1, NAGS, ASS1 and ASL were highly expressed, and the expression levels were almost similar to those in PHH. The test of urea synthesis ability also showed that the urea production produced by hepatocyte organoids in culture medium was almost the same as those by PHH. Finally, drug metabolism ability was tested, and the inducers rifampicin and omeprazole were added to induce the expression of phase I metabolizing enzymes. The expression levels of CYP3A4, CYP2C9, CYP1A1 and CYP1B1 in hepatocyte organoids were close to or even higher than those in PHH after the induction with inducers, and metabolic function of CYP3A4, CYP2C9 and CYP1A2 were tested using the kit, and the results shows that drug metabolism levels are higher compared to those in hepatocyte organoids without the treatment with the inducers, further confirming that the expression levels of phase I metabolizing enzymes were significantly increased after the induction.

In addition, human hepatocytes in liver are polarized under normal physiological conditions, that is, it has an apical membrane and a basal membrane to achieve directional absorption and release of nutrients and substances synthesized by hepatocytes. In addition to having specific functions, organoids also have structural features or characteristics that are major difference from cell spheroids. Mature hepatocyte organoids under such 3D suspension conditions are also polarized. Through immunofluorescent staining analysis, it has been shown that the apical membrane markers ZO1, MDR1 and Factin are internally located in hepatocyte organoids, while the basal membrane marker NTCP is expressed on the outside of hepatocyte organoids, that is, on the side of the basal membrane. These results have demonstrated that mature hepatocyte organoids induced by the present disclosure have significant polarization characteristics, which are similar to the in vivo feature. In addition, through CDFDA staining analysis, it had been shown that mature hepatocyte organoids induced under such 3D suspension conditions have significant bile canalicular distribution, which is particularly important for subsequent applications of hepatocyte organoids, such as drug screening and the development of viral hepatitis models. These features of polarized hepatocyte organoids were quite different from unpolarized hepatocyte spheroids.

The above results show that after long-term culture, HB organoids still have the ability to be induced to differentiate towards mature and polarized functional hepatocyte organoids with liver function comparable to PHH. This technology provides the support and assurance for large-scale production of functional hepatocytes.

2. The development of disease model (hepatocyte lipid accumulation model) using mature hepatocyte organoid included the steps as the following:

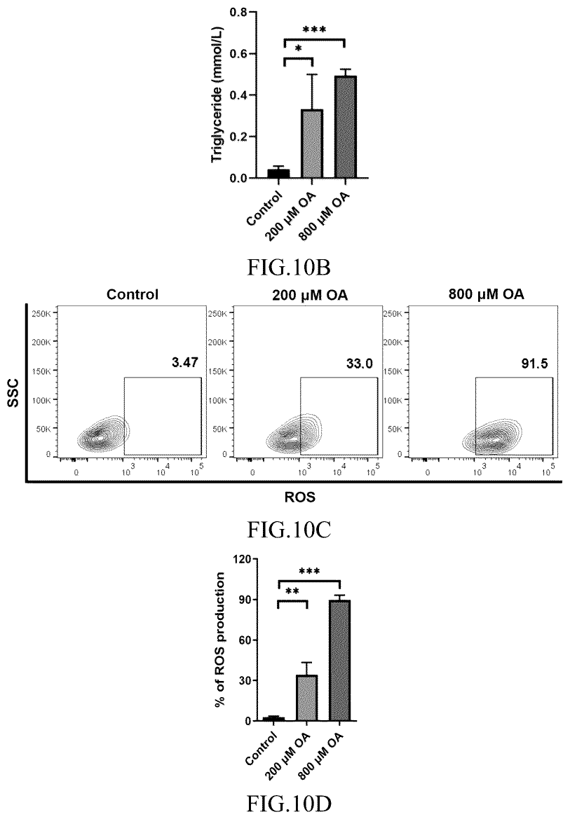

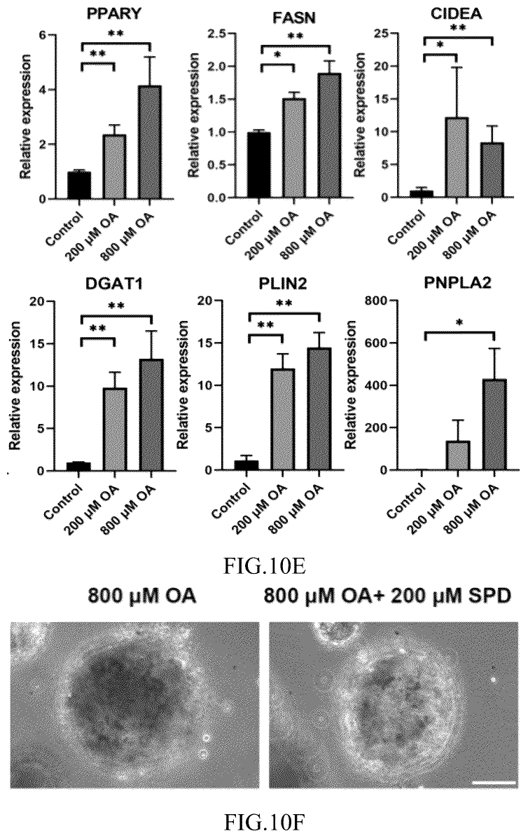

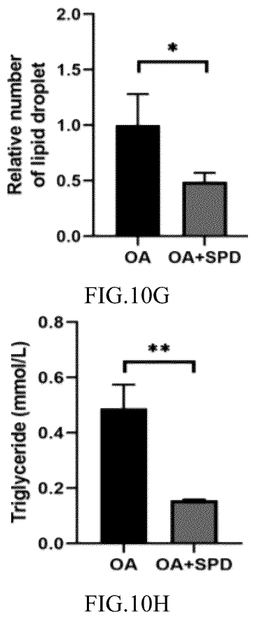

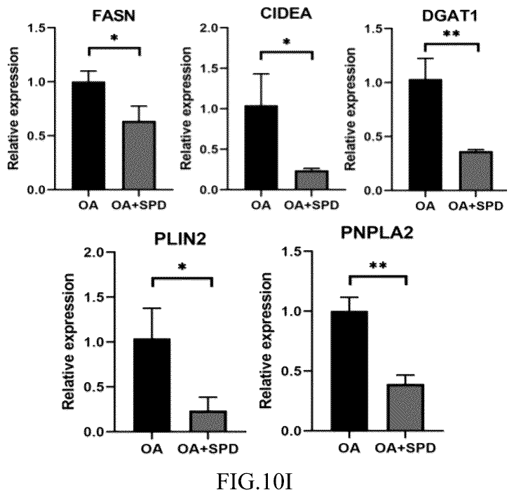

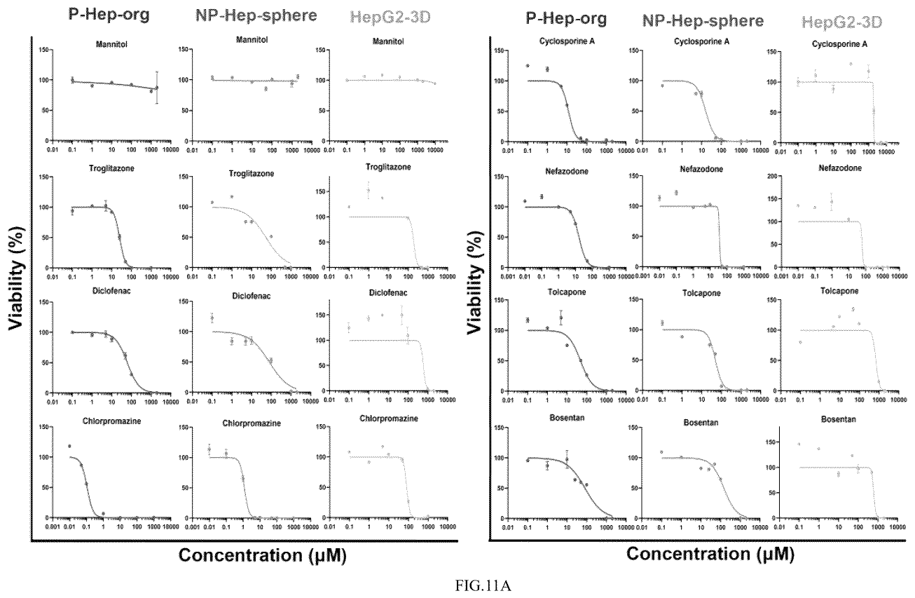

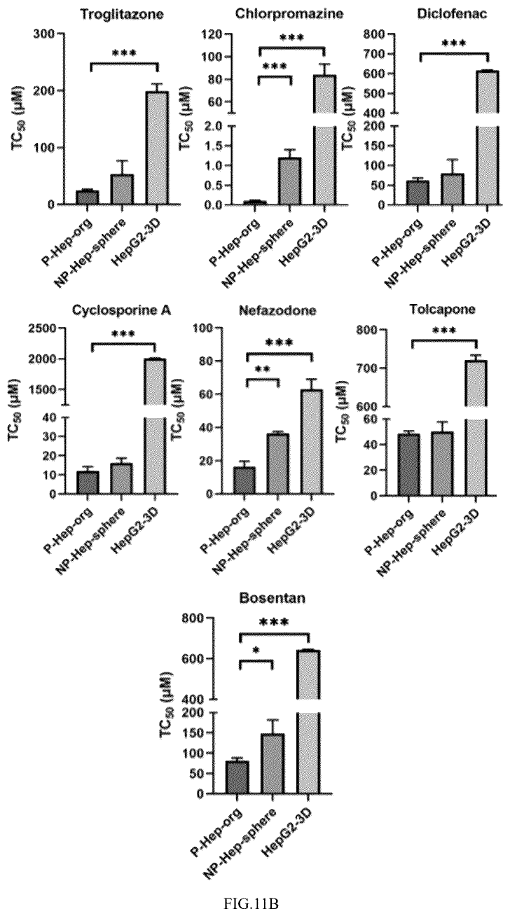

After mature hepatocyte organoids were differentiated from long-term cultured HB organoids (the lipid accumulation model could be developed 6 days after the differentiation), oleic acid (OA) was used with the concentrations at 200 and 800 μM to induce the lipid accumulation model. Two days after the treatment with OA, using oil red staining, it has been shown that the content of lipid droplets in hepatocyte organoids is gradually increased with the increase of OA concentration, indicating that hepatocyte organoids have undergone the steatosis. Afterwards, flow cytometry was used to detect the levels of intracellular oxidative stress ROS, and the results showed that the levels of intracellular ROS were increased with the addition of OA, indicating that oxidative stress had been generated in hepatocyte organoids. Then the levels of intracellular triglyceride were measured using the kit, and the results also showed that the levels of the intracellular triglyceride also were increase with the increase of OA concentration in the medium, indicating the occurrence of lipid accumulation in hepatocytes. The gene expression in hepatocyte organoids after modeling was detected by qPCR, and the results also showed that the expression levels of lipid accumulation related genes FASN and PLIN2 were significantly increased, indicating successful development of the lipid accumulation model. Next spermidine (SPD), a drug that alleviates lipid accumulation in hepatocytes, was used to conduct the rescue. SPD was added into the medium at 200 μM at the same time during the treatment with OA. Two days after the treatment with SPD, the results show that the relative number of lipid droplets in SPD-treated group is significantly reduced, and the expression levels of genes related to lipid accumulation are also decreased (FIG. 10A-10I), indicating that the lipid accumulation model developed by polarized hepatocyte organoids prepared in this Example, can successfully induce corresponding symptoms of the disease and can be relieved by drug treatment. The above results lay the foundation for the subsequent applications of polarized hepatocyte organoids in the development of drugs related to hepatocyte lipid accumulation and the treatment of lipid accumulation.

### Example 6: Drug Screening, Drug Development and Toxicology Analysis

The development of the method for hepatocyte drug screening/drug toxicology analysis using mature polarized hepatocyte organoids.

Polarized hepatocyte organoids (could be used in drug screening models 6 days after the maturation) after the maturation from the differentiation were collected, and distributed into ultra-low attachment 96-well culture plates using the pipette, about 10-20 organoids were seeded in each well, then different drugs could be employed for toxicology analysis. In this Example, toxicology analysis was performed on 8 kinds of drugs (Mannitol, Troglitazone, Chlorpromazine, Diclofenac, Cyclosporine A, Nefazodone, Tolcapone, Bosentan) to detect the ability of polarized hepatocyte organoids to predict the toxicity of each drug during drug screening/toxicology analysis. During the toxicology analysis, at least 7 different concentrations were selected for each drug, and wells in triplicate were performed for each concentration. Forty-eight hours after drug treatment, cell viabilities are detected using cell viability detection kit and IC50 curves were drawn. Finally, IC50 value of each drug for each cell type was calculated. In addition to polarized hepatocyte organoids, non-polarized hepatocyte spheroids and HepG2 cells (hepatoma cell lines, commonly used for hepatocyte drug screening) spheroids were also simultaneously used for drug toxicology analysis.

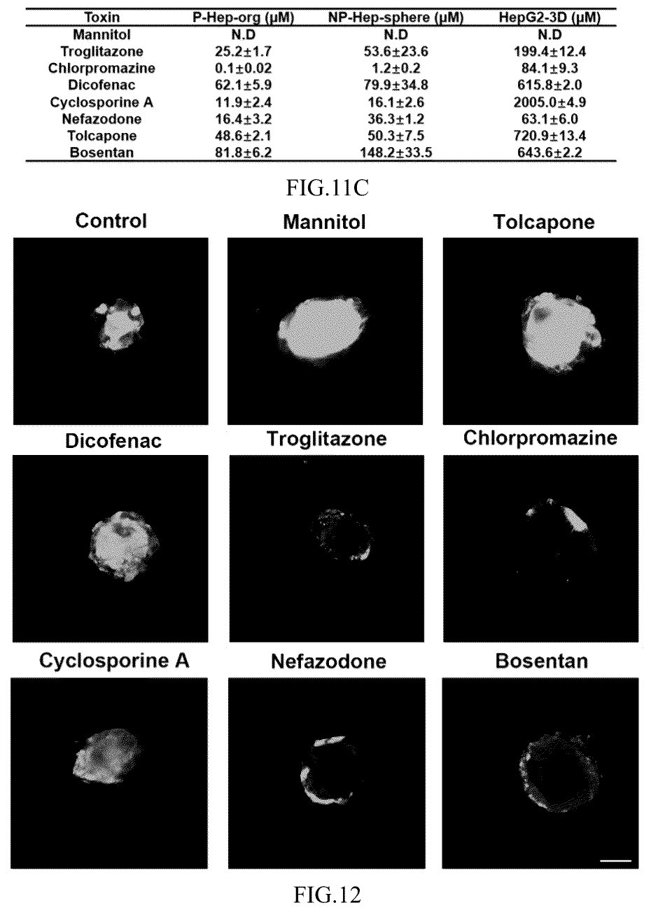

The final results show that for 8 different drugs, except for Mannitol which is no toxic to hepatocytes, polarized hepatocyte organoids had showed the strongest sensitivity to other drugs, with the lowest IC50 value. While HepG2 cells, a hepatoma cell line, often used for drug screening, had showed strong resistance to most drugs and had the highest IC50 value. These results indicate that if HepG2 cells are used in cell experiments for drug toxicity at early stage of drug development, IC50 value obtained under this circumstance does not precisely predict drug toxicity to cells and will not be applied in subsequent animal experiments and clinical studies. The sensitivity of polarized hepatocyte organoids to drugs can provide precise prediction on drug toxicity and quick reflection on the occurrence of drug-induced liver injury, laying the foundation for subsequent drug development. In addition, CDFDA staining was used to test the appearance of the cholestasis in polarized hepatocyte organoids which are treated by these drugs at IC25 values of each drug for 48 hours, and then the occurrence of the cholestasis after drug treatment was evaluated by whether fluorescent CDFDA product is transported into bile canaliculi through apical protein. The results show that, as reported, Mannitol, Tolcapone and Dicofenac do not induce the cholestasis, while other drugs cause the cholestasis with various degree, and result in the inability of CDFDA to enter the interior of hepatocyte organoids, indicating the damages on hepatocytes and polarization state to loss the bile canaliculi structure by drugs. These results demonstrate that the prepared polarized hepatocyte organoids can properly predict and reflect the consequence on the loss of hepatocytes caused by the drug (FIG. 11A-11C, FIG. 12).

### Example 7: Hepatocyte Organoids were Used to Treat Animals with Acute Liver Failure

A method for applying mature polarized hepatocyte organoids to treat mice with acute liver failure.

Mouse model with acute liver failure was developed using immunodeficient mice of FRG strain: two days before cell transplantation, thioacetamide (TAA) with the concentration of 600 mg/ml was intraperitoneally injected according to the body weight of each mouse. One day before cell transplantation, TAA with the concentration of 1200 mg/ml was intraperitoneally injected again according to the body weight of each mouse. If mice cannot be treated immediately at this time, they will normally die due to liver failure within 3-4 days. After the treatment with TAA injection, mice in the experimental group were transplanted with polarized hepatocyte organoids under the renal capsules 18 hours after the injection, while mice in the control group were received the same surgery, but injected with PBS into the renal capsule. After the treatments of the transplantation, mice were observed every day, the survival rate of mice was calculated, and the surviving mice were evaluated 7 days after the treatments.

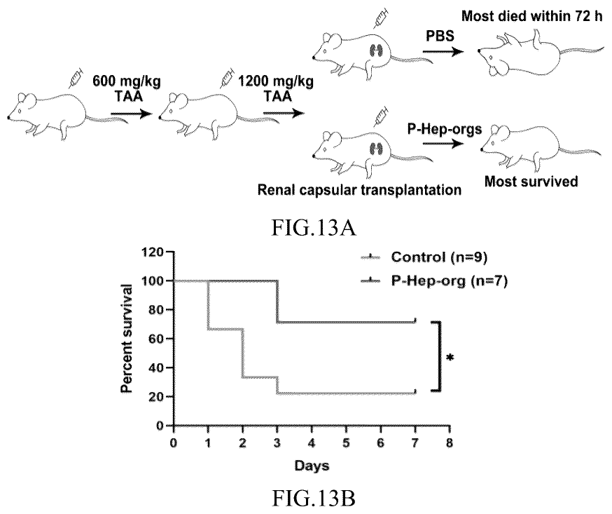

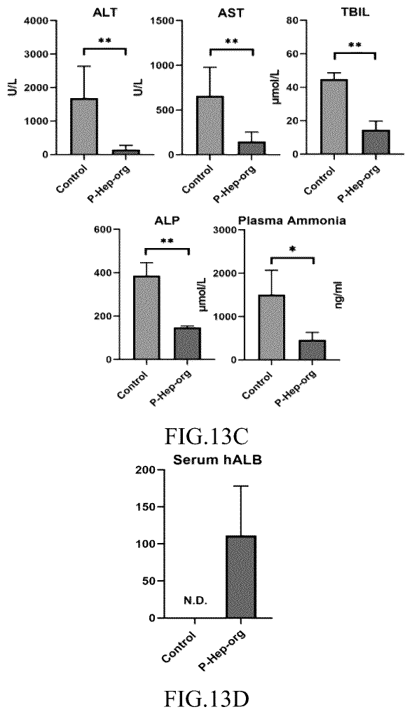

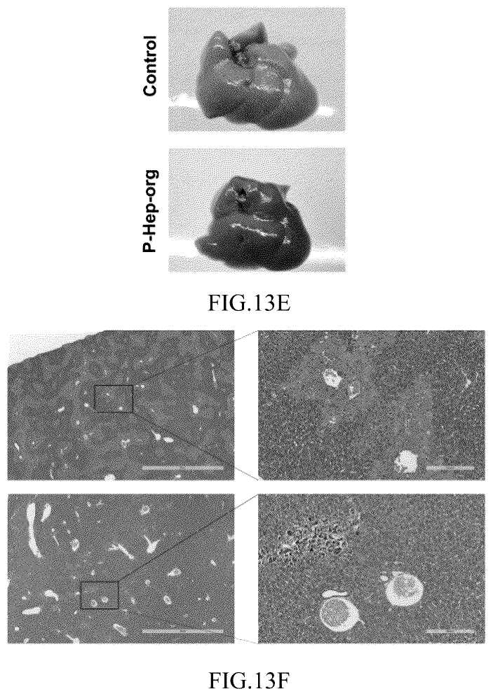

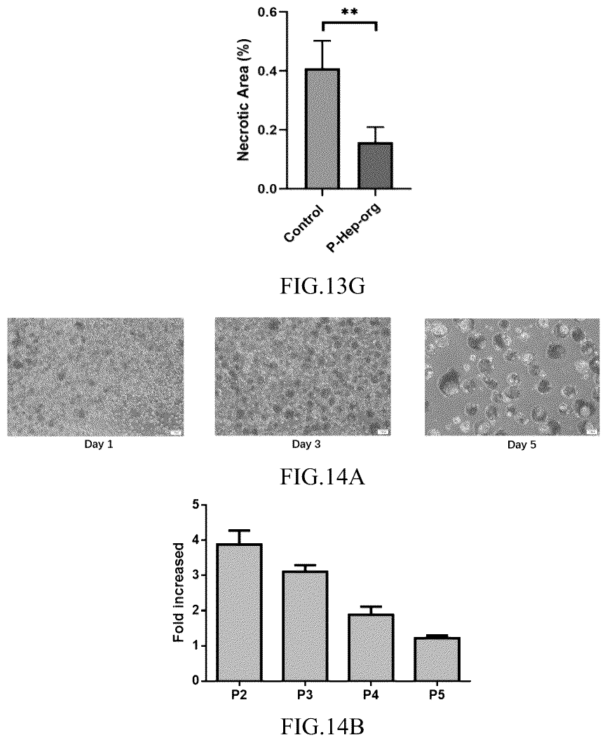

The results show that the survival rate of mice transplanted with polarized hepatocyte organoids is 71% at day 7 after the transplantation, while it is only 22% in mice of the control group. There is a significant difference on the survival rate between the two groups, indicating that polarized hepatocyte organoids can save the lives of mice with acute liver failure, and also indicating that polarized hepatocyte organoids can also perform liver functions in vivo. Liver damage indicators ALT, AST, TBIL, and ALP, as well as blood ammonia, were measured, and the results had showed that compared with the control group, the levels of these indicators were significantly reduced in mice of the experimental group, indicating those mice were able to recover faster from liver damage. Seven days after the treatment of the transplantation, mouse liver organs were removed and photographed from each group of mice. It had been shown that the appearance of the livers from mice transplanted with polarized hepatocyte organoids was very similar to that of normal mice, while the livers of mice from the control group obviously had liver necrosis areas (FIG. 13A-13G).

The above results show that polarized hepatocyte organoids not only have mature hepatocyte-related functions in vitro, but can also perform the functions of hepatocytes in mice and save the lives of mice with acute liver failure. These results have demonstrated that hepatocyte organoids can be used for hepatocyte cell transplantation and for cell sources of bioartificial liver device.

### Comparative Example 1

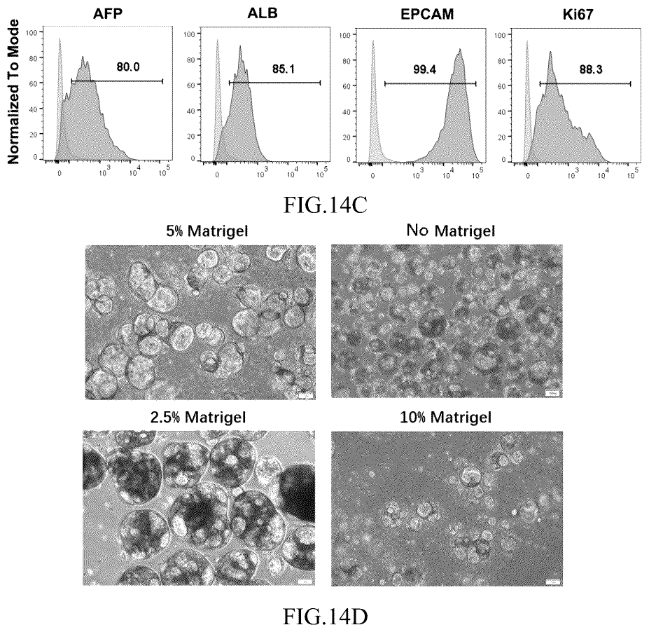

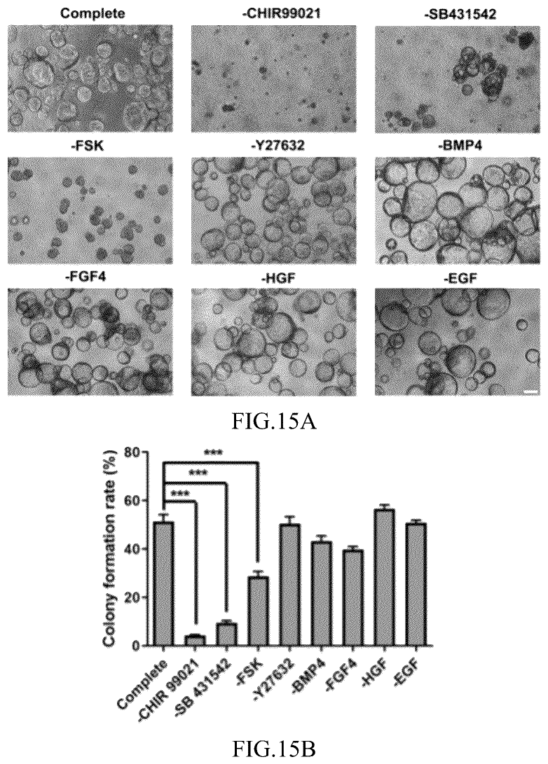

The medium used for the culture at the stage of the expansion of HB organoids was: IMDM medium supplemented with 10% FBS, 1×ITS, 1×NEAA, 1×GlutaMAX, 10 mM nicotinamide, 5 μM CHIR99021, 10 μM SB431542, 10 M FSK, 20 ng/mL FGF-4, 20 ng/mL BMP4, 20 ng/mL EGF and 5% Matrigel, in which Matrigel was crucial to this system, since Matrigel could provide extracellular matrix to support HB organoids for long-term culture under 3D suspension culture conditions. If Matrigel was removed from the system, cells would not be able to be cultured for a long time and could not be expanded efficiently, and the shape of cell spheroids no longer resemble the hollow structure with single layer as hepatocyte organoids. Instead, there were many vacuoles in the middle of solid hepatocyte spheroids, as shown in FIG. 14A. Under these conditions without Matrigel, cells gradually lose the capacity to expand, and were unable to continue to expand after passage 5, as shown in FIG. 14B. Flow cytometry analysis results show that cells could still maintain the expression of characteristic markers of hepatoblasts after only Matrigel was removed in the system, as shown in FIG. 14C, however, this system could not achieve the purpose of large-scale production of hepatocytes due to its inability to continue to support the expansion of cells. At the same time, the concentration of Matrigel at 5% is also very critical. As shown in FIG. 14D, at day 5 after the passage, under the conditions with 5% Matrigel, most HB organoids were formed as single, full and hollow organoids, while in 2.5% Matrigel group, HB organoids were multivesicular and spherical, very irregular, and difficult to directly induce into mature hepatocyte organoids. In 10% Matrigel group, the size of HB organoids was significantly smaller, indicating that under this condition, cell proliferation was slower than those in 5% Matrigel group. Compared with published methods in other literature, current protocols for hepatic organoids culture are restrictively dependent on hanging-drop Matrigel with high concentration to form microdroplets containing Matrigel and cells, which requires 50%-100% Matrigel to support organoid growth, making it difficult to be truly and subsequently applied in large-scale production^{[5, 6]}. Thus it can be seen that 5% Matrigel is essential for the large-scale expansion system of hepatoblasts established in the present disclosure.
5. Wang, S., et al., Human ESC-derived expandable hepatic organoids enable therapeutic liver repopulation and pathophysiological modeling of alcoholic liver injury. Cell Res, 2019. 29(12): p. 1009-1026.
6. Mun, S. J., et al., Generation of expandable human pluripotent stem cell-derived hepatocyte-like liver organoids. J Hepatol, 2019. 71(5): p. 970-985.

### Comparative Example 2

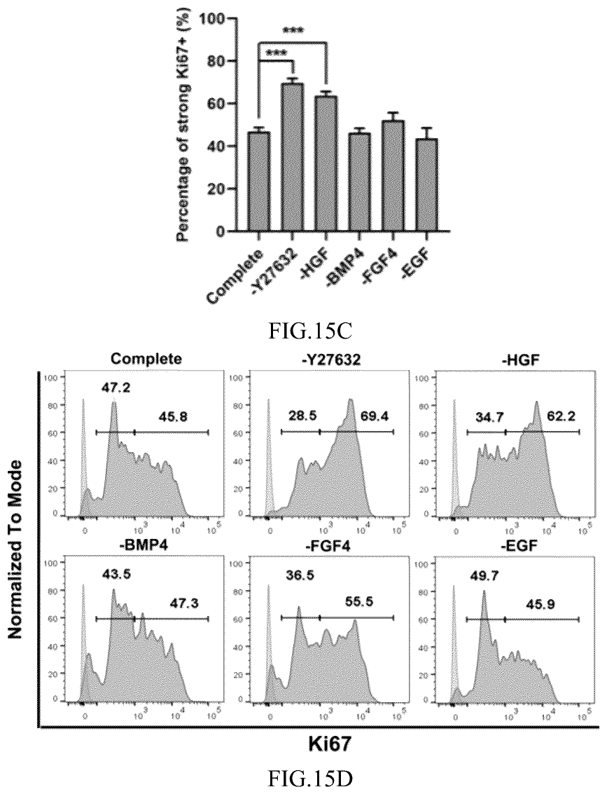

Based on the related-signaling pathways affecting the regeneration/proliferation/expansion of hepatoblasts in vivo and in vitro, the development of the medium formula for HB organoid expansion was to initially select small molecules or cytokines corresponding to the pathways to mimic the process of hepatic progenitor regeneration, and finally the combination of small molecules with cytokines had been established. Through comprehensive investigation on a large amount of literature, it had been shown that Wnt, TGFβ, cAMP, Activin, EGF and HGF pathways were related to the regeneration of hepatoblasts. Therefore, the combination with small molecules and cytokines initially selected for the expansion medium of hepatic progenitors had included CHIR99021, SB431542, FSK, Y27632, FGF-4, BMP4, HGF and EGF, this combination was found to be able to support long-term culture of hepatoblasts under 3D suspension conditions. Afterwards, the step-down method through removing factors one by one from the combination had been used to verify which small molecule/cytokine was essential for the long-term culture of hepatoblasts. The experimental results are shown in FIG. 15A-15D. First of all, from the morphological changes of cell spheroids, after removing CHIR99021 and FSK, cell spheroids had been unable to form hollow organoid-like structures, which was significantly different from those in the complete medium group. Secondly, from the analysis on the efficiency of cloning formation after inoculating cells, after removing CHIR99021, SB431542 and FSK, the efficiency of cloning formation had been significantly reduced, indicating that these three small molecules are essential for this expansion system, however, removing any one of these three cytokines will have little effect on both the morphologies of cell spheroids and the efficiency of cloning formation. Finally, flow cytometry had been used to detect the expression levels of cell proliferation marker Ki67. In addition to CHIR99021 and SB431542 which have been previously confirmed to be essential, the results show that compared with the expression levels of Ki67 in the complete medium group, Ki67 expression level had a higher expression peak after Y27632 and HGF were removed, indicating that the cell proliferation ability was more significant after removing these two small molecules/cytokines. Therefore, finally the expansion medium of HB organoid was determined as the fifth medium in Example 1: IMDM medium supplemented with 10 % w/w FBS, 1% w/w ITS, 1% w/w NEAA, 1% w/w GlutaMAX, 10 mM Nicotinamide, 5 μM CHIR99021, 10 M SB431542, 10 μM FSK, 20 ng/mL FGF-4, 20 ng/mL BMP4, 20 ng/mL EGF and 5% Matrigel.

The entire process of the present disclosure is carried out under 3D suspension culture conditions for the integration of culture and expansion of human pluripotent stem cells, the differentiation and sustainable culture as well as large-scale expansion of hepatoblast organoid, and large-scale differentiation of hepatocyte organoids. Compared with 2D culture conditions, 3D suspension culture conditions can truly achieve large-scale production of cells. However, previously reported long-term culture of hepatoblasts/organoids is only conducted under 2D culture conditions and cannot be applied to actual production. Under 3D suspension culture conditions in conjunction with employing 3D large-scale bioreactors, the integration of large-scale expansion and differentiation of cell production can be finally and efficiently realized. This is attributed to the combination of developed 3D suspension culture technology of human pluripotent stem cells and developed technology of 3D suspension differentiation with multiple stages as well as developed 3D suspension expansion technology in conjunction with using low concentration of Matrigel, all are established by the present disclosure.

The present disclosure can also simulate the development process and environment of hepatocytes in vivo. For hepatocytes, hepatocyte organoids formed under 3D suspension culture conditions can better simulate the in vivo 3D structure of the liver. At the same time, Matrigel induces the polarization of hepatocytes. Compared with hepatocytes cultured under 2D monolayer conditions, hepatocytes cultured under the present disclosure are more similar to the in vivo structural characteristics of liver tissue, and their liver functions are stronger. Hepatocyte organoids obtained in large scale are ultimately more suitable for various applications. This advantage is based on various technologies of 3D suspension culture and expansion established by the present disclosure.

The present disclosure uses the combination of low concentration of Matrigel and developed medium system for the expansion. Only 5% Matrigel is used as extracellular matrix to support large-scale expansion of hepatoblast organoids. The previous article has shown the importance of adding extracellular matrix, i.e. Matrigel, for long-term culture of hepatoblast organoids under 3D suspension culture conditions. This is very similar to 3D culture of organoids. However, high concentrations of Matrigel were used to form hanging-drops in current 3D culture of hepatocyte organoids, and then cells were inoculated for the culture. Hepatocyte organoids produced by this method also have the ability for long-term culture, but its ability of the expansion and efficiency of cloning formation are very low, thus it takes two weeks to passage cells. Moreover, using such high concentration of Matrigel cannot be combined with 3D scale bioreactor and cannot be truly applied to large-scale production of hepatocytes in practice, thus, this approach with high concentration of Matrigel is only performed in laboratory research. By using only 5% Matrigel as the extracellular matrix, the present disclosure can enable hepatoblast organoids to proliferate more efficiently under 3D suspension conditions, and is sufficient to support their long-term culture to 20 passages, which provides technical assurance for truly obtaining large-scale production of hepatocytes by combining with 3D large-scale bioreactors. This advantage is the results of extensively laborious practices and attempts, thus, the present disclosure successfully discovers that low concentration of 5% Matrigel can support the sustainable expansion of hepatoblast organoids under 3D suspension culture conditions, and this is the key for large-scale expansion of hepatoblast organoids and large-scale production of functional hepatocyte organoids.

The above specific embodiments have provided detailed descriptions of the present disclosure, but the present disclosure is not limited to the above embodiments. Various changes can be made within the knowledge scope of the ordinary skill in the art or without departing from the purpose of the present disclosure. In addition, the examples of the present disclosure and the features in the examples can be combined with each other without conflicts.

1. A medium composition, comprising a fifth medium, wherein the fifth medium comprises Matrigel, a TGFβ/ALK inhibitor, a GSK3β inhibitor and Forskolin (FSK).

2. The medium composition according to claim 1, wherein the medium composition further comprises a sixth medium, and the sixth medium comprises Matrigel and growth factors; the Matrigel in the sixth medium has a final concentration of 3-8 v/v %.

3. The medium composition according to claim 1, wherein the medium composition comprises a fourth medium, and the fourth medium comprises growth factors and BMP signaling pathway activators; the growth factors in the fourth medium comprise at least one of EGF, PDGF, FGF, HGF, IGF-1, IGF-II, LIF, NGF, oncostatin M, PDECGF, TGF-α, and VEGF; and the BMP signaling pathway activators in the fourth medium comprise at least one of BMP2, BMP4, SB4, SJ000291942, SJ000063181, SJ000370178, isoliquiritigenin, diosmetin, apigenin and biochanin.

4. The medium composition according to claim 1, wherein the medium composition further comprises a first medium, a second medium, and a third medium; and the first medium comprises Activin A and a GSK-3 inhibitor; the second medium comprises Activin A and a knockout serum replacement; the third medium comprises Activin A and a serum replacement.

5. (canceled)

6. (canceled)

7. (canceled)

8. A method for developing and/or continuously expanding hepatoblasthepatoblast organoids, comprising: culturing hepatoblasts by using the fifth medium in claim 1.

9. A method for developing mature hepatocyte organoids, comprising: culturing hepatoblast organoids by using the sixth medium in claim 2.

10. (canceled)

11. The medium composition according to claim 1, wherein the Matrigel in the fifth medium has a final concentration of 3-8 v/v %.

12. The medium composition according to claim 1, wherein the TGFβ/ALK inhibitor in the fifth medium has a final concentration of 5-20 μM.

13. The medium composition according to claim 1, wherein the GSK30 inhibitor in the fifth medium has a final concentration of 3-8 μM.

14. The medium composition according to claim 1, wherein the FSK in the fifth medium has a final concentration of 5-20 μM.

15. The medium composition according to claim 2, wherein the growth factors in the sixth medium comprise at least one of epidermal growth factor (EGF), platelet-derived growth factor (PDGF), fibroblast growth factor (FGF), hepatocyte growth factor (HGF), insulin-like growth factor-I (IGF-1), IGF-II, leukaemia inhibitory factor (LIF), nerve growth factor (NGF), oncostatin M (OSM), platelet-derived endothelial cell growth factor (PDECGF), transforming growth factor-α (TGF-α) and vascular endothelial cell growth factor (VEGF).

16. The medium composition according to claim 4, wherein the GSK-3 inhibitor in the first medium comprises at least one of B216763, TWS1 19, NP031112, SB216763, CHIR-98014, AZD2858, AZD1080, SB415286, LY2090314, and CHIR-99021.

17. A kit, comprising at least one of a first medium, a second medium, a third medium, a fourth medium, a fifth medium and a sixth medium,
wherein the first medium comprises Activin A and a GSK-3 inhibitor;
wherein the second medium comprises Activin A and a knockout serum replacement;
wherein the third medium comprises Activin A and a serum replacement;
wherein the fourth medium comprises growth factors and BMP signaling pathway activators; and the growth factors in the fourth medium comprise at least one of EGF PDGF, FGF, HGF, IGF-1, IGF-II LIF NGF, oncostatin M, PDECGF, TGF-α, and VEGF; and the BMP signaling pathway activators in the fourth medium comprise at least one of BMP2, BM4P4, SB4, SJ000291942, SJ000063 181, SJ000370178, isoliquiritigenin, diosmetin, apigenin and biochanin;
wherein the fifth medium comprises Matrigel, a TGFβ/ALK inhibitor, a GSK3β inhibitor and Forskolin (FSK); and
wherein the sixth medium comprises Matrigel and growth factors.

18. A method for preparing endoderm cells, hepatoblasts or hepatocytes, comprising: culturing and inducing human pluripotent stem cells to differentiate into endoderm cells using the kit according to claim 17 under 3D suspension culture conditions; or
culturing and inducing endoderm cells to differentiate into hepatoblast organoids using the kit according to claim 17 under 3D suspension culture conditions: or
culturing and inducing hepatoblast organoids to differentiate into hepatocyte organoids using the kit according to claim 17 under 3D suspension culture conditions.

19. A method for inducing stem cells to differentiate into endoderm cells, hepatoblasts or hepatocytes, comprising:
inducing the stem cells to differentiate into the endoderm cells using the kit according to claim 17 under 3D suspension culture conditions: or
inducing the endoderm cells to differentiate into the hepatoblast organoids using the kit according to claim 17 under 3D suspension culture conditions; or
inducing the hepatoblast organoids to differentiate into the hepatocyte organoids using the kit according to claim 17 under 3D suspension culture conditions.

20. The method according to claim 19, wherein the stein cells are human-derived stem cells with multidirectional differentiation potential.

21. The method according to claim 20, wherein the human-derived stem cells with multidirectional differentiation potential comprises human embryonic stem cells, human parthenogenetic stem cells, induced pluripotent stein cells, mesenchymal stem cells, adipose-derived stem cells and cord blood stem cells.

22. A method for treating a liver disease, comprising: transplanting the endoderm cells, the hepatoblasts or the hepatocytes obtained according to the method in claim 18 into the bodies of subjects in need.

23. A method for preparing a hepatocyte source for bioartificial liver device, comprising: inducing the stem cell to differentiate to the hepatocytes as cell source for bioartificial liver device using the kit according to claim 17 under 3D suspension culture conditions, wherein the hepatocyte source for bioartificial liver device comprises endoderm cells, hepatoblasts or hepatocytes.
